(19) United States
(12) Patent Application Publication
(10) Pub. No.: US 2011/0262909 A1
(43) Pub. Date: Oct. 27, 2011

(54) Genetic Markers for Horned and Polled Cattle and Related Methods

(75) Inventors: Edward J. Cargill, Cottleville, MO (US); Michael D. Grosz, Ellisville, MO (US); Nicholas J. Nissing, Chesterfield, MO (US)

(73) Assignee: Pfizer, Madison, NJ (US)

(21) Appl. No.: 12/681,034

(22) PCT Filed: Sep. 24, 2008
(86) PCT No.: PCT/US08/11024

§ 371 Date: Aug. 20, 2010

Related U.S. Application Data
(60) Provisional application No. 60/977,238, filed on Oct. 3, 2007.

(51) Int. Cl. C12Q 1/68

(52) U.S. Cl. 435/6.11

(57) ABSTRACT

Embodiments of the present invention provide methods for improving desirable animal traits including the horned/polled phenotype in bovine animals. Also provided are methods for determining a dairy animal's genotype with respect to multiple markers associated with polled, fitness and/or productivity traits. The invention also provides methods for selecting or allocating animals for predetermined uses such as progeny testing or nucleus herd breeding, for picking potential parent animals for breeding, and for producing improved progeny animals. Also provided are methods for identifying genetic markers associated with polled, fitness and/or productivity traits that are in allelic association with the SNPs disclosed herein.

### PRIORITY CLAIM

This application claims the benefit of U.S. Provisional Application Ser. No. 60/977,238 filed Oct. 3, 2007, which is herein incorporated by reference in its entirety.

### INCORPORATION OF SEQUENCE LISTING

A sequence listing is contained in the file named “Polled_v2_ST251_final.txt”, which is 55,296 bytes (54 Kilobytes) (measured in MS-Windows XP) and which comprises 148 sequences and was created on Sep. 19, 2008 is transmitted herewith and incorporated herein by reference.

### FIELD OF THE INVENTION

The invention relates to the enhancement of desirable characteristics in cattle. More specifically, it relates to the use of genetic markers in methods for improving cattle with respect to the horned/polled phenotype.

### BACKGROUND OF THE INVENTION

The future viability and competitiveness of the cattle, and specifically dairy, industry depends on continual improvement in animal phenotypes. Although many traits have some degree of underlying genetic variation in commercial cattle populations, the accuracy of selecting breeding animals with superior genetic merit for many of them is low due to low heritability or the inability to measure the trait cost effectively on the candidate animal. In addition, some traits such as the horned/polled phenotype are frequently difficult to characterize on an animal young enough to be useful. For example, nearly all young cattle are de-horned at an early age regardless of whether or not they are exhibiting horns, simply as a preventative treatment. Many of the animals dehorned may in fact be polled (genetically horn-less). Such procedures create significant difficulties in obtaining accurate phenotypes needed for traditional breeding methods. Furthermore, the low frequency associated with the polled phenotype suggests traditional breeding methods would be inefficient in creating a substantially polled herd or breed of bovines. Thus, the accuracy of conventional selection for these traits is moderate to low and the ability to make genetic change through selection is limited.

Genomics offers the potential for greater improvement through the discovery of genes, or genetic markers linked to genes, that account for genetic variation and can be used for more direct and accurate selection. The horned/polled phenotype has been studied by several groups, particularly in beef cattle, but an accurate test which works effectively in all populations has eluded all researchers. See for example Brenneman et al (1996); Drogemuller et al (2005); Georges et al (1993); Harlizius et al (1997); Long and Gregory (1978); Schmutz et al (1995); White and Ibsen (1936); Wunderlich et al (2006)

Cattle herds used for milk production around the world originate predominantly from the Holstein or Holstein-Friesian breeds which are known for high levels of production. Since Holstein germplasm has been sold and transported globally for several decades, the Holstein breed has effectively become one large global population held to relatively moderate inbreeding rates. As such, this population is of particular interest with respect to developing an accurate genetic test for identifying and predicting the horned/polled phenotype, as well as it's incorporation into breeding programs. Furthermore, the use of markers for the horned/polled phenotype can be combined with selection for other traits such as fitness and productivity traits by using a plurality of genetic markers.

### SUMMARY OF THE INVENTION

This section provides a non-exhaustive summary of the present invention.

Various embodiments of the invention provide methods for evaluating an animal's genotype at one or more positions in the animal's genome. In various aspects of these embodiments the animal's genotype is evaluated at positions within a segment of DNA (an allele) that contains at least one SNP selected from the SNPs described in the Tables and Sequence Listing of the present application.

Other embodiments of the invention provide methods for allocating animals for use according to their predicted breeding value for the horned/polled phenotype. Various aspects of this embodiment of the invention provide methods that comprise: a) analyzing the animal's genomic sequence at one or more polymorphisms (where the alleles analyzed each comprise at least one SNP) to determine the animal's genotype at each of those polymorphisms; b) analyzing the genotype determined for each polymorphism to determine which allele of the SNP is present; c) allocating the animal for use based on its genotype at one or more of the polymorphisms analyzed. Various aspects of this embodiment of the invention provide methods for allocating animals for use based on a favorable association between the animal's genotype, at one or more polymorphisms disclosed in the present application in Table 1, and the horned/polled phenotype. Alternatively, the methods provide for not allocating an animal for a certain use because it has one or more SNP alleles that are either associated with undesirable phenotypes or are not associated with desirable phenotypes.

Other embodiments of the invention provide methods for selecting animals for use in breeding to produce progeny. Various aspects of these methods comprise: a) determining the genotype of at least one potential parent animal at one or more locus/loci, where at least one of the loci analyzed contains an allele of a SNP selected from the group of SNPs described in the Tables. b) analyzing the determined genotype at one or more positions for at least one animal to determine which of the SNP alleles is present. c) correlating the analyzed allele(s) with the horned/polled phenotype. d) allocating at least one animal for use to produce progeny.

Other embodiments of the invention provide methods for producing offspring animals (progeny animals). Aspects of this embodiment of the invention provide methods that comprise: breeding an animal that has been selected for breeding by methods described herein to produce offspring. The offspring may be produced by purely natural methods or through the use of any appropriate technical means, including but not limited to: artificial insemination; embryo transfer (ET), multiple ovulation embryo transfer (MOET), in vitro fertilization (IVF), or any combination thereof.

Other embodiments of the invention provide for databases or groups of databases, each database comprising lists of the nucleic acid sequences, which lists include a plurality of the SNPs described in the Tables. Preferred aspects of this embodiment of the invention provide for databases comprising the sequences for 5 or more SNPs. Other aspects of these embodiments comprise methods for using a computer algorithm or algorithms that use one or more database(s), each database comprising a plurality of the SNPs described in the Tables to identify phenotypic traits associated with the inheritance of one or more alleles of the SNPs, and/or using such a database to aid in animal allocation.

Additional embodiments of the invention provide methods for identifying other genetic markers that are in allelic association with one or more of the SNPs described in the Tables.

Embodiments of the present invention include a method for allocating an animal for use according to the animal's predicted horned/polled phenotype, the method comprising: a) determining the animal's genotype at one or more locus/loci; wherein at least one locus contains a single nucleotide polymorphism (SNP), having at least two allelic variants; and wherein at least one SNP is selected from the SNPs described in Table 1; b) analyzing the determined genotype of at least one evaluated animal at one or more SNPs selected from the SNPs described in Table 1 to determine which allelic variant is present; c) correlating the identified allele with a horned or polled phenotype; and d. allocating the animal for use according to its determined genotype.

Embodiments of the invention also include a method for selecting a potential parent animal for breeding potential offspring, comprising: a) determining at least one potential parent animal's genotype at one or more genomic locus/loci; wherein at least one locus contains a single nucleotide polymorphism (SNP) that has at least two allelic variants, and wherein at least one SNP is selected from the SNPs described in Table 1; b) analyzing the determined genotype of at least one evaluated animal for one or more SNPs selected from the SNPs described in Table 1 to determine which allele is present; c) correlating the identified allele with a horned or polled phenotype; d) allocating at least one animal for breeding use based on its genotype.

Embodiments of the invention also include a method of producing progeny animals comprising: a) identifying at least one potential parent animal that has been allocating for breeding as described herein; b) producing progeny from the allocated animal through a process comprising: (i) natural breeding; (ii) artificial insemination; (iii) in vitro fertilization; and/or (iv) collecting semen/spermatozoa or at least one ovum from the animal and contacting it, respectively, with ovum/ova or semen/spermatozoa from a second animal to produce a conceptus by any means.

Embodiments of the invention also include a nucleic acid array for determining which alleles are present in a sample; wherein the array comprises 2 or more nucleic acid sequences capable of hybridizing, under stringent conditions, with at least 1 or more SNPs selected from the group consisting of the SNPs described by Table 1.

Embodiments of the invention also include a method of determining which alleles are present in an animal comprising: a) providing an array comprising at least one nucleic acid sequence capable of hybridizing under stringent conditions to a SNP selected from the group consisting of the SNPS described by Tables 1 & 2; b) determining an animal's genotype using said array; c) correlating at least one identified allele with a horned or polled phenotype; and d) allocating at least one animal for breeding use based on its genotype.

Embodiments of the invention also include a method of identifying a genetic marker associated with the horned/polled phenotype by identifying a genetic marker in allelic association with at least one SNP selected from the group of SNPs described in Table 1, the method comprising: a) identifying a genetic marker B1 suspected of being in allelic association with a marker A1 selected from the group of SNPs described in Table 1; b) determining whether A1 and B1 are in allelic association; wherein allelic association exists if r2>0.2 for Equation 1 for a population sample of at least 100 animals and wherein A1 represents an allele of a SNP described in Table 1; B1 represents a genetic marker at another locus; f(A1B1) denotes frequency of having both A1 and B1; f(A1) is the frequency of A1 in the population; and f(B1) is the frequency of B1 in a population.

Embodiments of the invention also include a method for allocating an animal for use according to the animal's predicted horned/polled phenotype, the method comprising: a) determining the animal's genotype at one or more locus/loci; wherein at least one locus contains a genetic marker, having at least two allelic variants; and wherein at least one genetic marker is located in a gene described in Table 3; b) analyzing the determined genotype of at least one evaluated animal at one or more SNPs loci within a gene described in table 3 to determine which allelic variant is present; c)correlating the identified allele with a horned or polled phenotype; and c. allocating the animal for use according to its determined genotype.

Various embodiments of the invention also include methods of analysis that comprises whole-genome analysis.

Embodiments of the invention also comprise a method for selecting a potential parent animal for breeding potential offspring: a) determining at least one potential parent animal's genotype at one or more genomic locus/loci; wherein at least one loci contains a genetic markers located within a gene described in Table 3; b) analyzing the determined genotype of at least one evaluated animal for one or more genetic markers to determine which allele is present; c) correlating the identified allele with a horned or polled phenotype; and d) allocating at least one animal for breeding use based on its genotype.

Embodiments of the present invention comprise a method of producing progeny animals comprising: a) identifying at least one potential parent animal that has been allocating for breeding in accordance with any of the methods described herein, and b) producing progeny from the allocated animal through a process comprising: (i) natural breeding; (ii) artificial insemination; (iii) in vitro fertilization; and/or (iv) collecting semen/spermatozoa or at least one ovum from the animal and contacting it, respectively, with ovum/ova or semen/spermatozoa from a second animal to produce a conceptus by any means.

Embodiments of the invention also comprise a method of identifying a genetic marker associated with the horned or polled phenotype by identifying a genetic marker in allelic association with at least one marker located in a gene described in Table 3, the method comprising: a) identifying a genetic marker B1 suspected of being in allelic association with a marker A1 selected from the group of SNPs described in Table 3; b) determining whether A1 and B1 are in allelic association; wherein allelic association exists if r2>0.2 for Equation 1 for a population sample of at least 100 animals and wherein Al represents an allele of a SNP described in Table 3; B1 represents a genetic marker at another locus; f(A1B1) denotes frequency of having both A1 and B1; f(A1) is the frequency of A1 in the population; and f(B1) is the frequency of B1 in a population.

Embodiments of the invention also comprise a method of selecting a bovine animal using gene expression comprising the following steps: a) obtaining a sample from said animal; b) anal_{y}zing said sample for gene expression; and c) allocating said animal for use according to the analysis of gene expression; wherein said gene is selected from the genes described in Table 3.

Embodiments of the invention also comprise a nucleic acid array for determining which allele of at least 10 polymorphisms are present in a sample; wherein the array comprises one or more nucleic acid sequences capable of hybridizing, under stringent conditions, with one or more polymorphisms selected from the polymorphisms described in Tables 1 and 2.

Embodiments of the invention also comprise a method of evaluating an animal's genotype at one or more loci; wherein at least one locus comprises a polymorphism selected from the polymorphisms described in Table 1 and 2.

Embodiments of the invention also comprise isolated nucleic acid sequences comprising a sequence described in Tables 1 and/or 2 and/or the sequence listing. Preferred aspects of these embodiments of the invention provide for isolated nucleic acid molecules comprising 17 or more contiguous nucleotides from a sequence selected from the group of sequences described in Tables 1 and/or 2 and/or the Sequence Listing. Even more preferred aspects of these embodiments of the invention provide for isolated nucleic acid molecules comprising 18, 19, 20, 21, 22, 23, 24, 25, 26, 27, 28, 29, 30, 31, 32, 33, 34, 35, 36 or more contiguous nucleotides from a sequence selected from the group of sequences described in Tables 1 and/or 2 and/or the Sequence Listing. Most preferable among these isolated nucleic acid molecules are those that comprise the polymorphic nucleotide described for the sequences in Tables 1 and/or 2 and/or the Sequence Listing.

Various embodiments of the invention include a n isolated nucleic acid sequence comprising at least 17 contiguous nucleotides of a nucleic acid selected from the group of nucleic acids described in Tables 1 & 2 and the Sequence Listing; wherein the nucleic acid comprises at least one polymorphic nucleotide described in Tables 1 and 2 and the Sequence Listing. Other embodiments of the invention provide for isolated nucleic acid sequences capable of hybridizing under stringent conditions to any of the nucleic acid sequences described above.

Additional embodiments of the invention provide methods of measuring gene expression at the transcription level (i.e., messenger RNA, mRNA) for any of the genes listed in Table 2. Aspects of this embodiment of the invention provide methods that comprise: analyzing the mRNA expression of a gene listed in the Tables by methods described herein. The mRNA level of expression may be analyzed through the use of any appropriate technical means, including but not limited to: northern blot analysis, reverse transcription PCR (RT-PCR), quantitative PCR (QT-PCR), or any combination thereof.

Additional embodiments of the invention provide methods of measuring gene expression at the translation level (i.e., protein product) for any of the genes listed in the Tables. Aspects of this embodiment of the invention provide methods that comprise analyzing the protein expression and/or function of a gene listed in the Tables by methods described herein. The protein expression and/or function may be analyzed through the use of any appropriate technical means, including but not limited to: western blot analysis, polyclonal antibody-based tests, monoclonal antibody-based tests, protein electrophoresis, protein assays, microarrays, immunohistochemistry, competition assays, and enzyme assays. Enzyme-Linked ImmunoSorbent Assay (ELISA), lateral flow devices, or any combination thereof. Protein analysis and antibody based tests are well known in the art. Specific examples of protein-based analysis for bovine testing include WO03043524A2, US20060199235A1, WO06073447A2, and WO04059282A2.

Alternative embodiments of the invention may include methods of testing wherein the for horned/polled phenotype test is conducted in a rapid manner to allow for the selection of the animal for a specific purpose in a timely manner, such as through on-site testing using a genetic marker test or antibody-based test such as an ELISA.

Alternative embodiment of the present invention may also include the modification of expression of genes through the use of RNA interference (RNAi) technologies, as has been described in the art (Golding et al. 2006), for any of the genes listed in the Tables. Aspects of this embodiment of the invention provide methods that comprise: development or identification of microRNA, short hairpin RNA (shRNA), or silencing RNA (siRNA), expression of microRNA, shRNA, or siRNA in a vector, and delivery of the expression vector to a bovine. Modification of expression of genes using RNAi may be achieved through the use of any appropriate technical means, including but not limited to: chemical synthesis of microRNA, shRNA, or siRNA, expression vectors or viral vectors containing microRNA, shRNA, or siRNA, PCR-generated microRNA, shRNA, or siRNA expression cassettes and/or in vitro transcribed microRNA, shRNA, or siRNA, populations of microRNA, shRNA, or siRNA generated from RNase III or Dicer digested dsRNA, or any combination thereof.

Embodiments of the invention include methods of selecting wherein both males and females of the species are tested for the horned/polled phenotype. However, in some instances selection based on genotype information from only one gender may be acceptable.

In various embodiments of the invention, a male animal is tested for the polled phenotype and semen from the animal is collected and used for multiple breedings via artificial insemination. In a further preferred embodiment, a male animal is selected for the polled phenotype, semen is collected, the semen is then sorted or otherwise modified to bias the proportion of genders in the resulting progeny, and the gender-biased semen is used for breeding via artificial insemination.

### Definitions

The following definitions are provided to aid those skilled in the art to more readily understand and appreciate the full scope of the present invention. Nevertheless, as indicated in the definitions provided below, the definitions provided are not intended to be exclusive, unless so indicated. Rather, they are preferred definitions, provided to focus the skilled artisan on various illustrative embodiments of the invention.

As used herein the term “allelic association” preferably means: nonrandom deviation of f(AᵢBⱼ) from the product of f(Aᵢ) and f(Bⱼ), which is specifically defined by r²>0.2, where r² is measured from a reasonably large animal sample (e.g., ≧100) and defined as

$${r}^{2} = \frac{{[ f ( {A}_{1} {B}_{1} ) - f ( {A}_{1} ) f ( {B}_{1} ) ]}^{2}}{f ( {A}_{1} ) ( 1 - f ( {A}_{1} ) ) ( f ( {B}_{1} ) ( 1 - f ( {B}_{1} ) )} \qquad [ Equation 1 ]$$

where A₁ represents an allele at one locus, B₁ represents an allele at another locus; f(A₁B₁) denotes frequency of having both A₁ and B₁, f(A₁) is the frequency of A₁, f(B₁) is the frequency of B₁ in a population.

As used herein the terms “allocating animals for use” and “allocation for use” preferably mean deciding how an animal will be used within a herd or that it will be removed from the herd to achieve desired herd management goals. For example, an animal might be allocated for use as a breeding animal or allocated for sale as a non-breeding animal (e.g. allocated to animals intended to be sold for meat). In certain aspects of the invention, animals may be allocated for use in sub-groups within the breeding programs that have very specific goals (e.g. productivity or fitness). Accordingly, even within the group of animals allocated for breeding purposes, there may be more specific allocation for use to achieve more specific and/or specialized breeding goals.

As used herein the terms “polled” preferably refers to the phenotype of an animal which does not possess horns due to it's genotype, when evaluated in a species which may contain horns. Animals that are genetically predisposed to having horns but have been treated to remove or prevent growth of horns are not considered polled, even though they do not possess horns.

As used herein, the term “genetic marker” preferably refers to any stable and inherited variation in DNA that can be measured or detected by a suitable method. Genetic markers can be used to detect the presence of a specific genotype or phenotype other than itself, which is otherwise not measurable or very difficult to detect. Examples of genetic markers include, but are not limited to, Single Nucleotide Polymorphism (SNP), Restriction Fragment Length Polymorphism (RFLP), Amplified Fragment Length Polymorphism (AFLP), Copy Number Variation (CNV), Simple Sequence Repeat (SSR, also called microsatellite) and insertions/deletions.

As used herein the term “linkage disequilibrium” preferably means allelic association wherein A₁ and B₁ (as used in the above definition of allelic association) are present on the same chromosome.

As used herein the term “marker-assisted selection (MAS) preferably refers to the selection of animals on the basis of genetic marker information in possible combination with pedigree and phenotypic data.

As used herein the term “natural breeding” preferably refers to mating animals without human intervention in the fertilization process. That is, without the use of mechanical or technical methods such as artificial insemination or embryo transfer. The term does not refer to selection of the parent animals.

As used herein the term “predicted value” preferably refers to an estimate of an animal's breeding value or transmitting ability based on its genotype and pedigree.

As used herein the term “qualitative trait” is used to denote a trait that is controlled by a single gene or locus, which contributes the complete effect on the trait. The observations on qualitative traits are binary (i.e., yes or no).

As used herein the term “reproductive material” includes, but is not limited to semen, spermatozoa, ova, and zygote(s).

As used herein the term “single nucleotide polymorphism” or “SNP” refers to a location in an animal's genome that is polymorphic within the population. That is, within the population some individual animals have one type of base at that position, while others have a different base. For example, a SNP might refer to a location in the genome where some animals have a “G” in their DNA sequence, while others have a “T”, or as each individual contains two copies of DNA sequence a single animal may have one “G” and one “T” in their DNA sequence.

As used herein the terms “hybridization under stringent conditions” and “stringent hybridization conditions” preferably mean conditions under which a “probe” will hybridize to its target sequence to a detectably greater degree than to other sequences (e.g., at least 5-fold over background). Stringent conditions are target-sequence-dependent and will differ depending on the structure of the polynucleotide. By controlling the stringency of the hybridization and/or washing conditions, target sequences that are 100% complementary to the probe can be identified (homologous probing). Alternatively, stringency conditions can be adjusted to allow some mismatching in sequences so that lower degrees of similarity are detected (heterologous probing).

Typically, stringent conditions will be those in which the salt concentration is less than about 1.5 M Na ion, typically about 0.01 to 1.0 M Na ion concentration (or other salts) at pH 7.0 to 8.3 and the temperature is at least about 30° C. for short probes (e.g., 10 to 50 nucleotides) and at least about 60° C. for long probes (e.g., greater than 50 nucleotides). Stringency may also be adjusted with the addition of destabilizing agents such as formamide. Exemplary low stringency conditions include hybridization with a buffer solution of 30 to 35% formamide, 1 M NaCl, 1% SDS (sodium dodecyl sulphate) at 37° C., and a wash in 1× to 2×SSC (20×SSC=3.0 M NaCl/0.3 M trisodium citrate) at 50 to 55° C. Exemplary moderate stringency conditions include hybridization in 40 to 45% formamide, 1 M NaCl, 1% SDS at 37° C., and a wash in 0.5× to 1×SSC at 55 to 60° C. Exemplary high stringency conditions include hybridization in 50% formamide, 1 M NaCl, 1% SDS at 37° C., and a wash in 0.1×SSC at 60 to 65° C. The duration of hybridization is generally less than about 24 hours, usually about 4 to about 12 hours.

Specificity is typically the function of post-hybridization washes, the critical factors being the ionic strength and temperature of the final wash solution. For DNA-DNA hybrids, the thermal melting point (Tₘ) can be approximated from the equation of Meinkoth and Wahl (1984) Anal. Biochem. 138:267-284: Tₘ=81.5° C.+16.6 (log M) +0.41 (% GC) −0.61 (% form) −500/L; where M is the molarity of monovalent cations, % GC is the percentage of guanine and cytosine nucleotides in the DNA, % form is the percentage of formamide in the hybridization solution, and L is the length of the hybrid in base pairs. The Tₘ is the temperature (under defined ionic strength and pH) at which 50% of a complementary target sequence hybridizes to a perfectly matched probe. Tₘ is reduced by about 1° C. for each 1% of mismatching; thus, Tₘ, hybridization, and/or wash conditions can be adjusted to hybridize to sequences of the desired identity. For example, if sequences with 90% identity are sought, the Tₘ can be decreased 10° C. Generally, stringent conditions are selected to be about 5° C. lower than the Tₘ for the specific sequence and its complement at a defined ionic strength and pH. However, highly stringent conditions can utilize a hybridization and/or wash at 1, 2, 3, or 4° C. lower than the thermal melting point (Tₘ); moderately stringent conditions can utilize a hybridization and/or wash at 6, 7, 8, 9, or 10° C. lower than the thermal melting point (Tₘ); low stringency conditions can utilize a hybridization and/or wash at 11, 12, 13, 14, 15, or 20° C. lower than the thermal melting point (Tₘ). Using the equation, hybridization and wash compositions, and desired Tₘ, those of ordinary skill will understand that variations in the stringency of hybridization and/or wash solutions are inherently described. If the desired degree of mismatching results in a Tₘ of less than 45° C. (aqueous solution) or 32° C. (formamide solution), it is preferred to increase the SSC concentration so that a higher temperature can be used. An extensive guide to the hybridization of nucleic acids is found in Tijssen (1993) Laboratory Techniques in Biochemistry and Molecular Biology-Hybridization with Nucleic Acid Probes, Part I, Chapter 2 (Elsevier, N.Y.); and Ausubel et al., eds. (1995) Current Protocols in Molecular Biology, Chapter 2 (Greene Publishing and Wiley-Interscience, New York). See also Sambrook et al. (1989) Molecular Cloning: A Laboratory Manual (2d ed., Cold Spring Harbor Laboratory Press, Plainview, N.Y.).

### Illustrative Embodiments of the Invention

Various embodiments of the present invention provide methods for evaluating an animal's (especially a dairy animal's) genotype at one or more positions in the animal's genome. Aspects of these embodiments of the invention provide methods that comprise determining the animal's genomic sequence at one or more locations (loci) that contain single nucleotide polymorphisms (SNPs). Specifically, the invention provides methods for evaluating an animal's genotype by determining which of two or more alleles for the SNP are present for each of one or more SNPs selected from the group consisting of the SNPs described in the Tables of the instant application.

In certain aspects of these embodiments the animal's genotype is evaluated to determine which allele is present for 1 or more SNPs selected from the SNPs described in Table 1.

Various embodiments of the invention include a method for allocating an animal for use according to the animal's predicted horned/polled phenotype, the method comprising: a) determining the animal's genotype at one or more locus/loci; wherein at least one locus contains a single nucleotide polymorphism (SNP), having at least two allelic variants; and wherein at least one SNP is selected from the SNPs described in Table 1; b) analyzing the determined genotype of at least one evaluated animal at one or more SNPs selected from the SNPs described in Table 1 to determine which allelic variant is present; c) correlating the identified allele with a horned or polled phenotype; and allocating the animal for use according to its determined genotype.

Other embodiments of the invention include methods wherein the animal's genotype is evaluated at two or more loci that contain SNPs selected from the SNPs described in Table 1. Other embodiments include methods wherein the animal's genotype is evaluated at two or more loci, including at least one that contains SNPs selected from the SNPs described in Table 1. Other embodiments include methods wherein the animal's genotype is evaluated at one or more SNPs selected from the SNPs located on Table 1 and wherein the animal's genotype is evaluated at one or more SNPs selected from the SNPs located on Table 2. Other embodiments include methods wherein the animal's genotype is evaluated at 10 or more SNPs, including at least one SNP selected from the SNPs described in Table 1. Other embodiments include methods wherein at least one evaluated SNP is associated with fitness. Other embodiments include methods wherein at least one evaluated SNP is associated with productivity. Other embodiments include methods that comprise whole-genome analysis.

Various embodiments of the invention include a method for selecting a potential parent animal for breeding potential offspring comprising: a) determining at least one potential parent animal's genotype at one or more genomic locus/loci; wherein at least one locus contains a single nucleotide polymorphism (SNP) that has at least two allelic variants, and wherein at least one SNP is selected from the SNPs described in Table 1; b) analyzing the determined genotype of at least one evaluated animal for one or more SNPs selected from the SNPs described in Table 1 to determine which allele is present; c) correlating the identified allele with a horned or polled phenotype; d) allocating at least one animal for breeding use based on its genotype.

Other embodiments of the invention include methods wherein the potential parent animal's genotype is evaluated at 1 or more loci that contain SNPs selected from the SNPs described in Table 1, and wherein the potential parent animal's genotype is evaluated at one or more loci that contain SNPs selected from the SNPs described in Table 2. Other embodiments include methods wherein the potential parent animal's genotype is evaluated at 2 or more loci that contain SNPs selected from the SNPs described in Table 1. Other embodiments include methods wherein the potential parent animal's genotype is evaluated at 10 or more loci, including at least one loci that contains a SNP selected from the SNPs described in Table 1. Other embodiments include methods wherein the potential parent animal's genotype is evaluated at 20 or more loci, including at least one loci that contain SNPs selected from the SNPs described in Table 1. Other embodiments include methods wherein the potential parent animal is selected to propagate the polled phenotype in the potential offspring. Other embodiments include methods wherein the potential parent animal is selected to propagate the horned phenotype in the potential offspring. Other embodiments include methods that comprise whole-genome analysis.

Various embodiments of the invention include a method of producing progeny animals comprising: a) identifying at least one potential parent animal that has been allocating for breeding by (i). determining the animal's genotype at one or more locus/loci; wherein at least one locus contains a single nucleotide polymorphism (SNP), having at least two allelic variants; and wherein at least one SNP is selected from the SNPs described in Table 1; (ii). analyzing the determined genotype of at least one evaluated animal at one or more SNPs selected from the SNPs described in Table 1 to determine which allelic variant is present; (iii). correlating the identified allele with a horned or polled phenotype; and (iv). allocating the animal for use according to its determined genotype; b) producing progeny from the allocated animal through a process comprising: (i) natural breeding; (ii) artificial insemination; (iii) in vitro fertilization; and/or (iv) collecting semen/spermatozoa or at least one ovum from the animal and contacting it, respectively, with ovum/ova or semen/spermatozoa from a second animal to produce a conceptus by any means.

Other embodiments of the invention include methods comprising producing progeny through natural breeding. Other embodiments include methods comprising producing offspring through artificial insemination, embryo transfer, and/or in vitro fertilization. Other embodiments include methods wherein the potential parent animal's genotype is evaluated at 1 or more loci that contain SNPs selected from the SNPs described in Table 1 and wherein the potential parent animal's genotype is evaluated at 1 or more loci that contain SNPs selected from the SNPs described in Table 2.

Other embodiments of the invention include methods wherein the potential parent animal's genotype is evaluated at two or more loci that contain SNPs selected from the SNPs described in Table 1. Other embodiments include methods wherein the potential parent animal's genotype is evaluated at 10 or more loci, including at least one loci that contains a SNP selected from the SNPs described in Table 1. Other embodiments include methods wherein the potential parent animal's genotype is evaluated at 20 or more loci, including at least one loci that contain a SNP selected from the SNPs described in Table 1. Other embodiments include methods wherein the potential parent animal is selected to propagate the polled phenotype in the progeny. Other embodiments include methods wherein the potential parent animal is selected to propagate the horned phenotype in the progeny. Other embodiments include methods that comprise whole-genome analysis.

Various embodiments of the invention include a nucleic acid array for determining which alleles are present in a sample; wherein the array comprises 2 or more nucleic acid sequences capable of hybridizing, under stringent conditions, with at least 1 or more SNPs selected from the group consisting of the SNPs described by Table 1. Other embodiments of the invention include methods wherein the array is capable of determining which allele is present for each of 2 or more SNPs selected from the group consisting of the SNPs described by Table 1. Other embodiments include methods wherein the array is capable of determining which allele is present for each of 10 or more SNPs, including at least one locus selected from the group consisting of the SNPs described by Table 1. Other embodiments include methods wherein the array is capable of determining which allele is present for each of 100 or more SNPs, including at least one loci selected from the group consisting of the SNPs described by Table 1.

Various embodiments of the invention include a method of determining which alleles are present in an animal comprising: providing an array as described in the preceding paragraph; determining an animal's genotype using said array; correlating at least one identified allele with a horned or polled phenotype; and allocating at least one animal for breeding use based on its genotype. Preferred embodiments include methods wherein said animal is selected for the polled phenotype.

Various embodiments of the invention include a method of identifying a genetic marker associated with the horned/polled phenotype by identifying a genetic marker in allelic association with at least one SNP selected from the group of SNPs described in Table 1, the method comprising: a) identifying a genetic marker B₁ suspected of being in allelic association with a marker A₁ selected from the group of SNPs described in Table 1; b) determining whether A₁ and B₁ are in allelic association; wherein allelic association exists if r²>0.2 for Equation 1 for a population sample of at least 100 animals and wherein Equation 1 is:

$${r}^{2} = \frac{{[ f ( {A}_{1} {B}_{1} ) - f ( {A}_{1} ) f ( {B}_{1} ) ]}^{2}}{f ( {A}_{1} ) ( 1 - f ( {A}_{1} ) ) ( f ( {B}_{1} ) ( 1 - f ( {B}_{1} ) )} \qquad [ Equation 1 ]$$

and wherein A₁ represents an allele of a SNP described in Table 1; B₁ represents a genetic marker at another locus; f(A₁B₁) denotes frequency of having both A₁ and B₁; f(A₁) is the frequency of A₁ in the population; and f(B₁) is the frequency of B₁ in a population.

Other embodiments of the invention include methods wherein the genetic marker B₁ is a SNP. Other embodiments include methods wherein the genetic marker identified is in linkage disequilibrium with at least one SNP selected from the group of SNPs described in Table 1. Other embodiments include methods wherein B₁ is a causal mutation underlying a quantitative or qualitative trait locus related to the horned/polled phenotype. Preferred embodiments include methods wherein r²>0.5. More preferred embodiments include methods wherein r²>0.9.

Other embodiments of the invention include methods for identifying a genetic marker in allelic association with a SNP associated with the polled phenotype. Other embodiments include methods wherein the genetic marker is in linkage disequilibrium with a SNP associated with the polled phenotype. Other embodiments include methods wherein the identified genetic marker is a causative mutation.

Various embodiments of the invention include a method for allocating an animal for use according to the animal's predicted horned/polled phenotype, the method comprising: a) determining the animal's genotype at one or more locus/loci; wherein at least one locus contains a genetic marker, having at least two allelic variants; and wherein at least one genetic marker is located in a gene described in Table 3; b) analyzing the determined genotype of at least one evaluated animal at one or more SNPs loci within a gene described in table 3 to determine which allelic variant is present; c) correlating the identified allele with a horned or polled phenotype; and allocating the animal for use according to its determined genotype.

Other embodiments of the invention include methods wherein the animal's genotype is evaluated at two or more loci that contain a genetic marker located within a gene described in Table 3. Other embodiments of the invention include methods wherein the animal's genotype is evaluated at two or more loci, including at least one loci that contains a genetic marker located within a gene described in Table 3. Preferred embodiments include methods wherein the genetic marker is a polymorphism. More preferred embodiments include methods where said genetic marker is a single-nucleotide polymorphism (SNP).

Other embodiments of the invention include methods wherein the animal's genotype is evaluated at two or more loci, at least one of which contains a genetic marker located within a gene described in Table 3. Other embodiments include methods wherein at least one evaluated marker is associated with fitness. Other embodiments include methods wherein at least one evaluated marker is associated with productivity. Still other embodiments include methods that comprise whole-genome analysis.

Various embodiments of the invention include a method for selecting a potential parent animal for breeding potential offspring: a) determining at least one potential parent animal's genotype at one or more genomic locus/loci; wherein at least one loci contains a genetic markers located within a gene described in Table 3; b) analyzing the determined genotype of at least one evaluated animal for one or more genetic markers to determine which allele is present; c) correlating the identified allele with a horned or polled phenotype; and d. allocating at least one animal for breeding use based on its genotype.

Other embodiments of the invention include methods wherein said genetic marker is a polymorphism. Preferred embodiments include methods wherein said polymorphism is a single-nucleotide polymorphism (SNP). Other embodiments include methods wherein the potential parent animal's genotype is evaluated at two or more loci, at least one of which that contains a genetic markers located within a gene described in Table 3. Other embodiments include methods wherein the potential parent animal's genotype is evaluated at 10 or more loci, at least one of which that contains a genetic markers located within a gene described in Table 3. Preferred embodiments include methods wherein the potential parent animal is selected to propagate the polled phenotype in the potential offspring. Other embodiments include methods wherein the potential parent animal is selected to propagate the horned phenotype in the potential offspring. Additional embodiments include methods that comprise whole-genome analysis.

Various embodiments of the invention include a method of producing progeny animals comprising: a) identifying at least one potential parent animal that has been allocating for breeding by: (i) determining at least one potential parent animal's genotype at one or more genomic locus/loci; wherein at least one loci contains a genetic markers located within a gene described in Table 3; (ii) analyzing the determined genotype of at least one evaluated animal for one or more genetic markers to determine which allele is present; (iii) correlating the identified allele with a horned or polled phenotype; and (iv) allocating at least one animal for breeding use based on its genotype; b) producing progeny from the allocated animal through a process comprising: (i) natural breeding; (ii) artificial insemination; (iii) in vitro fertilization; and/or (iv) collecting semen/spermatozoa or at least one ovum from the animal and contacting it, respectively, with ovum/ova or semen/spermatozoa from a second animal to produce a conceptus by any means.

Other embodiments of the invention include methods comprising producing progeny through natural breeding. Other embodiments include methods comprising producing offspring through artificial insemination, embryo transfer, and/or in vitro fertilization. Preferred embodiments include methods wherein said genetic marker is a polymorphism. More preferred embodiments include methods where said genetic marker is a single-nucleotide polymorphism (SNP).

Various embodiments of the invention include a method of identifying a genetic marker associated with the horned or polled phenotype by identifying a genetic marker in allelic association with at least one marker located in a gene described in Table 3, the method comprising: a) identifying a genetic marker B₁ suspected of being in allelic association with a marker A₁ selected from the group of SNPs described in Table 3; b) determining whether A₁ and B₁ are in allelic association; wherein allelic association exists if r²>0.2 for Equation 1 for a population sample of at least 100 animals and wherein Equation 1 is:

$${r}^{2} = \frac{{[ f ( {A}_{1} {B}_{1} ) - f ( {A}_{1} ) f ( {B}_{1} ) ]}^{2}}{f ( {A}_{1} ) ( 1 - f ( {A}_{1} ) ) ( f ( {B}_{1} ) ( 1 - f ( {B}_{1} ) )} \qquad [ Equation 1 ]$$

and wherein A₁ represents an allele of a SNP described in Table 3; B₁ represents a genetic marker at another locus; f(A₁B₁) denotes frequency of having both A₁ and B₁; f(A₁) is the frequency of A₁ in the population; and f(B₁) is the frequency of B₁ in a population.

Other embodiments of the invention include methods wherein the genetic marker B₁ is a SNP. Other embodiments include methods wherein the genetic marker identified is in linkage disequilibrium with at least one SNP located within a gene described in Table 3. Other embodiments include methods wherein B₁ is a causal mutation underlying a quantitative or qualitative trait locus related to the horned/polled phenotype. Preferred embodiments include methods wherein r²>0.5. More preferred embodiments include methods wherein r²>0.9. Preferred embodiments also include methods for identifying a genetic marker in allelic association with a SNP associated with the polled phenotype. Other embodiments include methods wherein the genetic marker is in linkage disequilibrium with a SNP associated with the polled phenotype. Other embodiments include methods wherein the identified genetic marker is a causative mutation.

Various embodiments of the invention include a method of selecting a bovine animal using gene expression comprising the following steps: a) obtaining a sample from said animal; b) analyzing said sample for gene expression; and c) allocating said animal for use according to the analysis of gene expression; wherein said gene is selected from the genes described in Table 3.

Other embodiments of the invention include methods wherein said gene expression is measured at the transcription level through analysis of mRNA. Other embodiments include methods wherein said gene expression is measured at the translational level through analysis of protein content of said sample. Other embodiments include methods wherein the protein content is analyzed using a test selected from the group consisting of western blot analysis, polyclonal antibody based tests, monoclonal antibody based tests, protein electrophoresis, protein assays, microarrays, immunohistochemistry, competition assays, enzyme assays, and an Enzyme-Linked ImmunoSorbent Assay (ELISA). Other embodiments include methods wherein the protein content is analyzed using a lateral flow test comprising antibodies. Other embodiments include methods wherein the protein content is analyzed using an Enzyme-Linked ImmunoSorbent Assay (ELISA).

Various embodiments of the invention include a nucleic acid array for determining which allele of at least 10 polymorphisms are present in a sample; wherein the array comprises one or more nucleic acid sequences capable of hybridizing, under stringent conditions, with one or more polymorphisms selected from the polymorphisms described in Tables 1 and 2.

Other embodiments of the invention include arrays wherein the array comprises two or more polymorphisms selected from the polymorphisms described in Tables 1 and 2. Other embodiments include arrays wherein the array comprises five or more polymorphisms selected from the polymorphisms described in Tables 1 and 2. Other embodiments include arrays wherein the array comprises 10 or more polymorphisms selected from the polymorphisms described in Tables 1 and 2.

Various embodiments of the invention include a method of evaluating an animal's genotype at one or more loci; wherein at least one locus comprises a polymorphism selected from the polymorphisms described in Table 1 and 2.

Other embodiments of the invention include methods comprising evaluating an animal's genotype at 10 or more geriomic locus/loc. Other embodiments include methods wherein the animal's genotype is evaluated at 10 or more loci, wherein at least two loci comprise a polymorphism selected from the polymorphisms described in Table 3 and the Sequence Listing. Other embodiments include methods wherein the animal's genotype is evaluated at 20 or more loci wherein at least two loci comprise a polymorphism selected from the polymorphisms described in Table 3 and the Sequence Listing. Other embodiments include methods that comprise whole-genome analysis.

Various embodiments of the invention include a n isolated nucleic acid sequence comprising at least 17 contiguous nucleotides of a nucleic acid selected from the group of nucleic acids described in Tables 1 and 2 and the Sequence Listing; wherein the nucleic acid comprises at least one polymorphic nucleotide described in Tables 1 and 2 and the Sequence Listing. Various embodiments of the invention also include an isolated nucleic acid sequence comprising a sequence capable of hybridizing under stringent conditions to nucleic acids sequences described in Tables 1 and 2 and the sequence listing.

Embodiments of the invention also comprise isolated nucleic acid sequences comprising a sequence described in Tables 1 and/or 2 and/or the sequence listing. Preferred aspects of these embodiments of the invention provide for isolated nucleic acid molecules comprising 17 or more contiguous nucleotides from a sequence selected from the group of sequences described in Tables 1 and/or 2 and/or the Sequence Listing. Even more preferred aspects of these embodiments of the invention provide for isolated nucleic acid molecules comprising 18, 19, 20, 21, 22, 23, 24, 25, 26, 27, 28, 29, 30, 31, 32, 33, 34, 35, 36 or more contiguous nucleotides from a sequence selected from the group of sequences described in Tables 1 and/or 2 and/or the Sequence Listing. Most preferable among these isolated nucleic acid molecules are those that comprise the polymorphic nucleotide described for the sequences in Tables 1 and/or 2 and/or the Sequence Listing.

Other embodiments of the invention provide for isolated nucleic acid sequences capable of hybridizing under stringent conditions to any of the nucleic acid sequence described in above.

In any embodiment of the invention the genomic sequence at the SNP locus may be determined by any means compatible with the present invention. Suitable means are well known to those skilled in the art and include, but are not limited to direct sequencing, sequencing by synthesis, primer extension, Matrix Assisted Laser Desorption/Ionization-Time Of Flight (MALDI-TOF) mass spectrometry, polymerase chain reaction, restriction fragment length polymorphism, microarray/multiplex array systems (e.g. those available from Affymetrix, Santa Clara, Calif.), and allele-specific hybridization.

Other embodiments of the invention provide methods for allocating animals for subsequent use (e.g. to be used as sires or dams or to be sold for meat or dairy purposes) according to their predicted value for polled, productivity, or fitness traits. Various aspects of this embodiment of the invention comprise determining at least one animal's genotype for at least one SNP selected from the group of SNPs described in the Tables (methods for determining animals' genotypes for one or more SNPs are described supra). Thus, the animal's allocation for use may be determined based on its genotype at one or more, 5 or more, 25 or more, 50 or more, or 100 or more markers, including at least one of the SNPs described in the Tables.

The instant invention provides embodiments where analysis of the genotypes of the SNPs described in Table 1 is the only analysis done. Other embodiments provide methods where analysis of the SNPs disclosed herein is combined with any other desired type of genomic or phenotypic analysis (e.g. analysis of any genetic markers beyond those disclosed in the instant invention). Moreover, the SNPs analyzed may be selected from those SNPs only associated with the horned/polled phenotype, or the analysis may be done for SNPs selected from any desired combination of horned/polled, fitness, and/or productivity traits.

According to various aspects of these embodiments of the invention, once the animal's genetic sequence for the selected SNP(s) have been determined, this information is evaluated to determine which allele of the SNP is present for at least one of the selected SNPs. Preferably the animal's allelic complement for all of the determined SNPs is evaluated. Finally, the animal is allocated for use based on its genotype for one or more of the SNP positions evaluated. Preferably, the allocation is made taking into account the animal's genotype at each of the SNPs evaluated, but its allocation may be based on any subset or subsets of the SNPs evaluated.

The allocation may be made based on any suitable criteria. For any SNP, a determination may be made as to whether one of the allele(s) is associated/correlated with desirable characteristics or associated with undesirable characteristics. This determination will often depend on breeding or herd management goals. Determination of which alleles are associated with desirable phenotypic characteristics can be made by any suitable means. Methods for determining these associations are well known in the art; moreover, aspects of the use of these methods are generally described in the EXAMPLES, below.

In various embodiments of the invention, phenotypic traits that may be associated with the SNPs of the current invention include, but are not limited to horned/polled, fitness, and productivity traits.

According to various aspects of this embodiment of the invention allocation for use of the animal may entail either positive selection for the animals having the desired genotype(s) (e.g. the animals with the desired genotypes are selected for breeding), negative selection of animals having undesirable genotypes (e.g. animals with an undesirable genotypes are culled from the herd), or any combination of these methods. According to preferred aspects of this embodiment of the invention animals identified as having SNP alleles associated with desirable phenotypes are allocated for use consistent with that phenotype (e.g. allocated for breeding based on phenotypes positively associated with polled). Alternatively, animals that do not have SNP genotypes that are positively correlated with the desired phenotype (or possess SNP alleles that are negatively correlated with that phenotype) are not allocated for the same use as those with a positive correlation for the trait.

Other embodiments of the invention provide methods for selecting potential parent animals (i.e., allocation for breeding) to yield offspring having the polled phenotype. Various aspects of this embodiment of the invention comprise determining at least one animal's genotype for at least one SNP selected from the group of SNPs described in Table 1. Furthermore, determination of whether and how an animal will be used as a potential parent animal may be based on its genotype at one or more, 10 or more, 25 or more, 50 or more, or 100 or more SNPS, including at least one of the SNPs described in Table 1. Moreover, as with other types of allocation for use, various aspects of these embodiments of the invention provide methods where the only analysis done is to genotype the animal for one or more of the SNPs described in Table 1. Other aspects of these embodiments provide methods where analysis of one or more SNPs disclosed herein is combined with any other desired genomic or phenotypic analysis (e.g. analysis of any genetic markers beyond those disclosed in the instant invention). Moreover, the SNP(s) analyzed may all be selected from those associated only with polled/horned or only with fitness or only with productivity. Conversely, the analysis may be done for SNPs selected from any desired combination of these or other traits.

According to various aspects of these embodiments of the invention, once the animal's genetic sequence at the site of the selected SNP(s) have been determined, this information is evaluated to determine which allele of the SNP is present for at least one of the selected SNPs. Preferably the animal's allelic complement for all of the sequenced SNPs is evaluated. Additionally, the animal's allelic complement is analyzed and correlated with the probability that the animal's progeny will express one or more phenotypic traits. Finally, the animal is allocated for breeding use based on its genotype for one or more of the SNP positions evaluated and the probability that it will pass the desired genotype(s)/allele(s) to its progeny. Preferably, the breeding allocation is made taking into account the animal's genotype at each of the SNPs evaluated. However, its breeding allocation may be based on any subset or subsets of the SNPs evaluated.

The breeding allocation may be made based on any suitable criteria. For example, breeding allocation may be made so as to increase the probability of enhancing a single certain desirable characteristic in a population, in preference to other characteristics, (e.g. the polled phenotype); alternatively, the selection may be made so as to generally maximize overall production based on a combination of traits. The allocations chosen are dependent on the breeding goals. Sub-categories falling within fitness, include, inter alia: daughter pregnancy rate (DPR), productive life (PL), and somatic cell score. Sub-categories falling within productivity include, inter alia: milk fat percentage, milk fat yield, total milk yield, milk protein percentage, and total milk protein.

Other embodiments of the instant invention provide methods for producing progeny animals. According to various aspects of this embodiment of the invention, the animals used to produce the progeny are those that have been allocated for breeding according to any of the embodiments of the current invention. Those using the animals to produce progeny may perform the necessary analysis or, alternatively, those producing the progeny may obtain animals that have been analyzed by another. The progeny may be produced by any appropriate means, including, but not limited to using: (i) natural breeding, (ii) artificial insemination, (iii) in vitro fertilization (IVF) or (iv) collecting semen/spermatozoa and/or at least one ovum from the animal and contacting it, respectively with ova/ovum or semen/spermatozoa from a second animal to produce a conceptus by any means.

According to preferred aspects of this embodiment of the invention the progeny are produced by a process comprising natural breeding. In other aspects of this embodiment the progeny are produced through a process comprising the use of standard artificial insemination (AI), in vitro fertilization, multiple ovulation embryo transfer (MOET), or any combination thereof.

Other embodiments of the invention provide for methods that comprise allocating an animal for breeding purposes and collecting/isolating genetic material from that animal: wherein genetic material includes but is not limited to: semen, spermatozoa, ovum, zygotes, blood, tissue, serum, DNA, and RNA.

It is understood that most efficient and effective use of the methods and information provided by the instant invention employ computer programs and/or electronically accessible databases that comprise all or a portion of the sequences disclosed in the instant application. Accordingly, the various embodiments of the instant invention provide for databases comprising all or a portion of the sequences corresponding to at least one SNP described in the Tables.

It is further understood that efficient analysis and use of the methods and information provided by the instant invention will employ the use of automated genotyping; particularly when large numbers of markers are evaluated. Any suitable method known in the art may be used to perform such genotyping, including, but not limited to the use of micro-arrays.

Other embodiments of the invention provide methods wherein one or more of the SNP sequence databases described herein are accessed by one or more computer-executable programs. Such methods include, but are not limited to, use of the databases by programs to analyze for an association between the SNP and a phenotypic trait, or other user-defined trait (e.g. traits measured using one or more metrics such as gene expression levels, protein expression levels, or chemical profiles), and programs used to allocate animals for breeding or market.

Other embodiments of the invention provide methods comprising collecting genetic material from an animal that has been allocated for breeding. Wherein the animal has been allocated for breeding by any of the methods disclosed as part of the instant invention.

Other embodiments of the invention provide for diagnostic kits or other diagnostic devices for determining which allele of a SNP is present in a sample; wherein the SNP(s) are selected from the group of SNPs described in the Tables. In various aspects of this embodiment of the invention, the kit or device provides reagents/instruments to facilitate a determination as to whether nucleic acid corresponding to the SNP is present. Such kit/or device may further facilitate a determination as to which allele of the SNP is present. In certain aspects of this embodiment of the invention the kit or device comprises at least one nucleic acid oligonucleotide suitable for DNA amplification (e.g. through polymerase chain reaction). In other aspects of the invention the kit or device comprises a purified nucleic acid fragment capable of specifically hybridizing, under stringent conditions, with at least one allele of at least one SNP described in the Tables. In preferred embodiments, the diagnostic kit contains a purified nucleic acid fragment capable of specifically hybridizing with at least one allele of at least one SNP described in Table 1.

In specific aspects of this embodiment of the invention the kit or device comprises at least one nucleic acid array (e.g. DNA micro-arrays) capable of determining which allele of one or more of the SNPs described in Table 1 is present in a sample. Preferred aspects of this embodiment of the invention provide DNA micro-arrays capable of simultaneously determining which allele is present in a sample for 1 or more SNPs. Preferably, the DNA micro-array is capable of determining which SNP allele is present in a sample for 5 or more, 25 or more, 50 or more, 100 or more, 200 or more, 500 or more, or 1000 or more SNPs. Methods for making such arrays are known to those skilled in the art and such arrays are commercially available (e.g. from Affymetrix, Santa Clara, Calif.).

Other embodiments of the instant invention provide methods of identifying genetic markers associated with the polled/horned phenotype that are in allelic association with one or more of the SNPs described in Table 1. According to various aspects of this embodiment of the invention the method comprises: (a) identifying a marker, B₁, that is suspected of being in allelic association with at least one marker, A₁, wherein A₁ is selected from the group of SNPs described in Table 1; (b) determining whether A₁ and B₁ are in allelic association; wherein allelic association is determined to exist if r²>0.2 for Equation 1 for a population sample of at least 100 animals and wherein Equation 1 is:

$${r}^{2} = \frac{{[ f ( {A}_{1} {B}_{1} ) - f ( {A}_{1} ) f ( {B}_{1} ) ]}^{2}}{f ( {A}_{1} ) ( 1 - f ( {A}_{1} ) ) ( f ( {B}_{1} ) ( 1 - f ( {B}_{1} ) )} \qquad [ Equation 1 ]$$

and wherein for Equation 1 A₁ represents an allele at one locus (A SNP described by Table 1); B₁ represents a genetic marker at another locus; f(A₁B₁) denotes frequency of having both A₁ and B₁; f(A₁) is the frequency of A₁ in the population, f(B₁) is the frequency of B₁ in the population. In preferred aspects of this embodiment of the invention B₁ is a SNP. Even more preferred aspects of this embodiment of the invention provide methods for identifying genetic markers in linkage disequilibrium with one or more SNPs selected from the group of SNPs described in Table 1. In other aspects of these embodiments of the invention the value of r² is greater than 0.5, greater than 0.7, or greater than 0.9.

Additional embodiments of the invention provide for genetic markers for the polled/horned phenotype that are in allelic association with one or more of the SNPs described in Table 1. Markers provided as part of this embodiment of the invention may be identified by any suitable means known to those of ordinary skill in the art. A marker falls within this embodiment of the invention if it is determined to be in allelic association with one or more of the SNPs described in Table 1 as defined by Equation 1, supra, where r² is greater than 0.2, greater than 0.5, greater than 0.7, or greater than 0.9. In preferred aspects of these embodiments of the invention the markers are in linkage disequilibrium with one or more of the SNPs described in Table 1.

Genetic markers for the polled/horned phenotype that are in allelic association with any of the SNPs described in Table 1 may be identified by any suitable means known to those skilled in the art. For example, a genomic library may be screened using a probe specific for any of the sequences of the SNPs described in the Tables. In this way clones comprising at least a portion of that sequence can be identified and then up to 300 kilobases of 3′ and/or 5′ flanking chromosomal sequence can be determined. For another example, the sequences of the SNPs described in the Tables may be used to query databases containing bovine DNA sequence data and hundreds, thousands, or millions of bases of 3′ and/or 5′ flanking chromosomal sequence can be determined. By any of these means, genetic markers in allelic association with the SNPs described in Table 1 will be identified.

Other embodiments of the present invention provide methods for identifying genes that may be associated with phenotypic variation. According to various aspects of these embodiments, the chromosomal location of a SNP associated with a particular phenotypic variation can be determined, by means well known to those skilled in the art. Once the chromosomal location is determined genes suspected to be involved with determination of the phenotype can be analyzed. Such genes may be identified by sequencing adjacent portions of the chromosome or by comparison with analogous section of the human genetic map (or known genetic maps for other species). An early example of the existence of clusters of conserved genes is reviewed in Womack (1987), where genes mapping to the same chromosome in one species were observed to map to the same chromosome in other, closely related, species. As mapping resolution improved, reports of the conservation of gene structure and order within conserved chromosomal regions were published (for example, Grosz et al, 1992). More recently, large scale radiation hybrid mapping and BAC sequence have yielded chromosome-scale comparative mapping predictions between human and bovine genomes (Everts-van der Wind et al., 2005), between human and porcine genomes (Yasue et al., 2006) and among vertebrate genomes (Demars et al., 2006).

### EXAMPLES

The following examples are included to demonstrate general embodiments of the invention. It should be appreciated by those of skill in the art that the techniques disclosed in the examples which follow represent techniques discovered by the inventors to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the invention.

All of the compositions and methods disclosed and claimed herein can be made and executed without undue experimentation in light of the present disclosure. While the compositions and methods of this invention have been described in terms of preferred embodiments, it will be apparent to those of skill in the art that variations may be applied without departing from the concept and scope of the invention.

### Example 1

### Identifying Markers Associated with the Horned/Polled Phenotype in Dairy Cattle

The polled mutation in Bos taurus, which is unknown, was localized to the proximal end of bovine chromosome 1 (BTA01) by Georges et al. (1993) utilizing microsatellite markers. More recent efforts to fine-map the polled locus have included additional microsatellite marker mapping (Schmutz et al. 1995; Brenneman et al. 1996; Harlizius et al. 1997; Drögemüller et al. 2005) and the creation of a BAC-based physical map of the polled region (Wunderlich et al. 2006). The location of the most proximal gene, ATP5O, and most distal gene, KRTAP8, of the polled region from these cited sources corresponds to approximately 0.6 Mb and 3.9 Mb respectively on the public bovine genome assembly version 3.1 (www.hgsc.bcm.tmc.edu/projects/bovine/).

The objective of this work was to identify single nucleotide polymorphisms (SNPs) associated with the polled trait by sequencing targeted regions of the proximal end of BTA01 on a discovery panel of polled and horned Holsteins.

### Discovery and Mapping Populations

All DNA samples were extracted from spermatozoa using the Qiagen Biosprint (Qiagen Inc., Valencia, Calif.) according to the manufacturer's protocol. Twenty-four Holstein bulls were utilized as a polymorphism discovery panel. Within the set of 24, pairs of animals are directly related as horned sires and polled sons, with the dams expected to be polled based on the reported phenotype of the animal. Semen samples from the 12 polled sons, were obtained from two dairy producers who specifically breed for the polled trait by utilizing polled females. To incorporate the industry elite genetics, top sires are bred to these polled females. Therefore, any polymorphism identified as concordant with the polled/horned trait would be homozygous for one allele in the 12 horned bulls and heterozygous (or infrequently homozygous for the second allele) in the 12 polled bulls. (Assuming that the allele frequency of the polled allele is 0.1, the probability of finding a homozygous polled son in this population can be calculated to be 10%).

### PCR

PCR primers were designed to target gene coding regions and regulatory elements (UTRs, putative promoters) including an average of 70 bp flanking sequence from target genes within the region. Optimal primer annealing temperatures were obtained by using gradient PCR thermocycling conditions of 15 minutes at 95° C., 35 cycles of 45 seconds at 94° C., 45 seconds of gradient temperatures starting at 55° to 66° across twelve sample wells, 45 seconds at 72° C., and 10 minutes 72° C. Once an optimal annealing temperature was found, each primer set was amplified for sequencing using standard thermocycling conditions of 15 minutes at 95° C. followed by 35 cycles of 45 seconds at 94° C., 45 seconds at optimal annealing temp, and 45 seconds at 72° C., with a final extension step of 10 minutes at 72° C. Concentrations for a 10 μl PCR volume (gradient and standard) were 5 ng/μl of genomic DNA, 0.5 μM of each primer (forward and reverse), 1× SIGMA JumpStart PCR Mix (Sigma-Aldrich Co., St. Louis, Mo.).

### Putative Regulatory Element Prediction

The on-line resource WWW Promoter Scan (www-bimas.cit.nih.gov/molbio/proscan/) was used to scan targeted gene introns and inter-genic sequences for predicted regulatory elements such as promoters and transcription factor binding sites. Identified putative regulatory elements were included with gene coding and regulatory regions (UTRs) for primer design and targeted polymorphism discovery.

### Sequencing

Sequencing was carried out using the method described below and all sequencing was done in both forward and reverse directions. A 10 μl standard PCR was performed, of which 5 μl was visualized by agarose gel electrophoresis for confirmation of amplification and the remaining 5 μl purified using the EXO-SAP-IT PCR Product Clean-up (USB corporation, Cleveland, Ohio) according to the manufacturer's protocol. Direct sequencing of purified PCR products was conducted in 9 μl reaction volume of 7 μl of purified PCR product and 2 μl of 10 μM primer, both forward or reverse, and resolved on an ABI 3730×1 Automated Sequencer (Applied Biosystems, Foster City, Calif.). Forward and reverse sequences were generated for each DNA sample. Sequence trace alignment and polymorphism detection was carried out using recent versions of Phred/Phrap (Ewing et al. 1998, Ewing and Green 1998) and Consed (Gordon et al, 1998).

SNPs discovered through the above described sequencing efforts were analyzed for genotypes matching that expected if the SNPs are associated with/causal to the horned/polled phenotype. The expected genotypic profile is: a) all sires homozygous for one allele, and all sons either homozygous for the other allele (or possibly heterozygous). Those SNPs listed in Table 1 showed 100% concordance with the predicted genotypic profile, and thus showed association with the horned/polled phenotype.

### Example 2

### Use of Single Nucleotide Polymorphisms to Improve Offspring Traits

To improve the average genetic merit of a population for a chosen trait, one or more of the markers with significant association to that trait can be used in selection of breeding animals. In the case of each discovered locus, use of animals possessing a marker allele (or a haplotype of multiple marker alleles) in population-wide LD with a favorable phenotype will increase the breeding value of animals used in breeding, increase the frequency of that allele (and phenotype) in the population over time and thereby increase the average genetic merit of the population for that trait. This increased genetic merit can be disseminated to commercial populations for full realization of value.

For example, a DNA-testing program scheme could greatly change the frequency of the polled allele in a given population or semen product via the use of DNA markers for screening bulls as described herein. Testing semen from bulls within a progeny testing program would identify the genotype of the bull at the horned/polled locus. This information creates value because this knowledge influences market desirability of the semen product. Typically, a progeny testing program uses pedigree information and performance of relatives to select juvenile bulls as candidates for entry into the program. However, by adding horned/polled marker information, young bulls could be screened to identify those animals carrying (or homozygous for) the polled marker/allele. The use of these animals to create the next generation of animals would not only create more naturally polled animals (since polled is dominant), but would also increase the frequency of the polled allele in the population from which the next generation of parents will ultimately be selected.

Additionally, DNA samples from potential bull mothers and their male offspring could be screened with markers from Table 1, and bull-mother candidates with preferable genotypes can be contracted for matings to tested bulls. If superovulation and embryo transfer (ET) is employed, a set of 5-10 offspring could be produced per bull mother per flush procedure. Then the markers could again be used to select a polled male offspring as a candidate for the progeny test program, or a female offspring as a future bull mother.

The first step in using a SNP for estimation of breeding value and selection in the genetic nucleus (GN) is collection of DNA from all offspring that will be candidates for selection as breeders in the GN or as breeders in other commercial populations. One method is to capture shortly after birth a small bit of ear tissue, hair sample, or blood from each calf into a labeled (bar-coded) tube. Another method is to directly test semen from bulls available for breeding. The DNA extracted from this tissue can be used to assay an essentially unlimited number of SNP markers, including the honed/polled markers described in Table 1, and the results can be included in selection decisions before the animal reaches breeding age.

The markers described herein can be used in breeding schemes in combination with markers that are associated with phenotypic traits of economic relevance. One method for incorporating into selection decisions the markers (or marker haplotypes) determined to be in population-wide LD with valuable QTL alleles is based on classical quantitative genetics and selection index theory (Falconer and Mackay, 1996; Dekkers and Chakraborty, 2001). To estimate the effect of the marker in the population targeted for selection, a random sample of animals with phenotypic measurements for the trait of interest can be analyzed with a mixed animal model with the marker fitted as a fixed effect or as a covariate (regression of phenotype on number of allele copies). Results from either method of fitting marker effects can be used to derive the allele substitution effects, and in turn the breeding value of the marker:

α₁=q[[a+d(q−p) [Equation 3]

α₂=−p[a+d(q−p)] [Equation 4]

α=a+d(q−p) [Equation 5]

g_{A1A1}=2(α₁) [Equation 6]

g_{A1A2}=(α₁)+(α₂) [Equation 7]

g_{A2A2}=2(α²) [Equation 8]

where α₁ and α₂ are the average effects of alleles 1 and 2, respectively; α is the average effect of allele substitution; p and q are the frequencies in the population of alleles 1 and 2, respectively; a and d are additive and dominance effects, respectively; g_{A1A1}, g_{A1A2} and g_{A2A2} are the (marker) breeding values for animals with marker genotypes A1A1, A1A2 and A2A2, respectively. The total trait breeding value for an animal is the sum of breeding values for each marker (or haplotype) considered and the residual polygenic breeding value:

EBVᵢⱼ=Σĝⱼ+Ûᵢ [Equation 9]

where EBVᵢⱼ is the Estimated Trait Breeding Value for the i^{th} animal, Σĝⱼ is the marker breeding value summed from j=1 to n where n is the total number of markers (haplotypes) under consideration, and Ûᵢ is the polygenic breeding value for the i^{th} animal after fitting the marker genotype(s).

These methods can readily be extended to estimate breeding values for selection candidates for multiple traits, the breeding value for each trait including information from multiple markers (haplotypes), all within the context of selection index theory and specific breeding objectives that set the relative importance of each trait. Other methods also exist for optimizing marker information in estimation of breeding values for multiple traits, including random models that account for recombination between markers and QTL (e.g., Fernando and Grossman, 1989), and the potential inclusion of all discovered marker information in whole-genome selection (Meuwissen et al., Genetics 2001). Through any of these methods, the markers reported herein could be determined to be in population-wide LD with valuable QTL alleles and may be used to provide greater accuracy of selection, greater rate of genetic improvement, and greater value accumulation in the dairy industry.

### Example 3

### Identification of a SNP

A nucleic acid sequence contains a SNP of the present invention if it comprises at least 20 consecutive nucleotides that include and/or are adjacent to a polymorphism described in Table 1, Table 2, and the Sequence Listing. Alternatively, a SNP of the present invention may be identified by a shorter stretch of consecutive nucleotides which include or are adjacent to a polymorphism which is described in Table 1, Table 2, and the Sequence Listing in instances where the shorter sequence of consecutive nucleotides is unique in the bovine genome. A SNP site is usually characterized by the consensus sequence in which the polymorphic site is contained, the position of the polymorphic site, and the various alleles at the polymorphic site. “Consensus sequence” means DNA sequence constructed as the consensus at each nucleotide position of a cluster of aligned sequences. Such clusters are often used to identify SNP and Indel (insertion/deletion) polymorphisms in alleles at a locus. Consensus sequence can be based on either strand of DNA at the locus, and states the nucleotide base of either one of each SNP allele in the locus and the nucleotide bases of all Indels in the locus, or both SNP alleles using degenerate code (IUPAC code: M for A or C; R for A or G; W for A or T; S for C or G; Y for C or T; K for G or T; V for A or C or G; H for A or C or T; D for A or G or T; B for C or G or T; N for A or C or G or T; Additional code that we use include I for “-”or A; O for “-” or C; E for “-” or G; L for “-” or T; where “-” means a deletion). Thus, although a consensus sequence may not be a copy of an actual DNA sequence, a consensus sequence is useful for precisely designing primers and probes for actual polymorphisms in the locus.

Such SNP have a nucleic acid sequence having at least 90% sequence identity, more preferably at least 95% or even more preferably for some alleles at least 98% and in many cases at least 99% sequence identity, to the sequence of the same number of nucleotides in either strand of a segment of animal DNA which includes or is adjacent to the polymorphism. The nucleotide sequence of one strand of such a segment of animal DNA may be found in a sequence in the group consisting of SEQ ID NO:1 through SEQ ED NO:148. It is understood by the very nature of polymorphisms that for at least some alleles there will be no identity at the polymorphic site itself. Thus, sequence identity can be determined for sequence that is exclusive of the polymorphism sequence.

Shown below are examples of public bovine SNPs that match each other: SNP ss38333809 was determined to be the same as ss38333810 because 41 bases (with the polymorphic site at the middle) from each sequence match one another perfectly (match length=41, identity=100%).

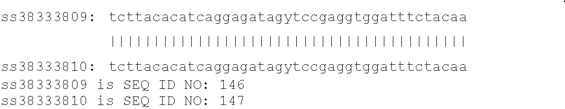

SNP ss38333809 was determined to be the same as ss38334335 because 41 bases (with the polymorphic site at the middle) from each sequence match one another at all bases except for one base (match length=41, identity =97%).

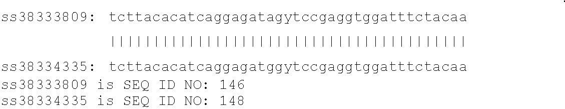

### REFERENCES

The references cited in this application, both above and below, are specifically incorporated herein by reference.

### Non-Patent Literature

Abdel-Azim, G and Freeman, A E, (2002) J. Daily Sci. 85:1869-1880.
Blott, S., Kim, J. J., Moisio, S., et al. (2003). Genetics 163: 253-266.
Brenneman R A, Davis S K, Sander J O, Burns B M, Wheeler T C, Turner J W, Taylor J F (1996). The polled locus maps to BTA1 in a Bos indicus×Bos taurus cross. Journal of Heredity 87:156-161.
Ciobanu, D C, Bastiaansen, J W M, Longergan, S M, Thomsen, H, Dekkers, J C M, Plastow, G S, and Rothschild, M F, (2004) J. Anim. Sci. 82:2829-39.
Cohen-Zinder, M. et al. (2005) Genome Res. 15:936-44.
Davis, G P and DeNise, S K, (1998) J. Anim. Sci. 76:2331-39.
Dekkers, J C M, and Chakraborty, R, (2001) J. Anim. Sci. 79:2975-90.
Demars J, Riquet J, Feve K, Gautier M, Morisson M, Demeure O, Renard C, Chardon P, Milan D. (2006), BMC Genomics, 24:7-13
Drögemüller C, Wöhlke A, Mömke S, Distl O (2005). Fine mapping of the polled locus to a 1-Mb region on bovine chromosome 1q12. Mammalian Genome 16:613-620.
Du and Hoeschele, (2000) Genetics 156:2051-62.
Ducrocq, V. 1987. An analysis of length of productive life in dairy cattle. Ph.D. Diss. Cornell Univ., Ithaca, N.Y.; Univ. Microfilms Int., Ann Arbor, Mich.
Everts-van der Wind A, Larkin D M, Green C A, Elliott J S, Olmstead C A, Chiu R, Schein J E, Marra M A, Womack J E, Lewin H A. (2005) Proc Natl Acad Sci USA, 20;102(51):18526-31.
Falconer, D S, and Mackay, T F C, (1996) Introduction to Quantitative Genetics. Harlow, UK: Longman.
Fernando, R, and Grossman, M, (1989) Marker assisted selection using best linear unbiased prediction. Genetics Selection Evolution 21:467-77.
Franco, M M, Antunes, R C, Silva, H D, and Goulart, L R (2005) J. Appl. Genet. 46(2): 195-200.
Georges M, Drinkwater R, King T, Mishra A, Moore S S, Nielsen D, Sargeant L S, Sorensen A, Steele M R, Zhao X, Womack J E, Hetzel J (1993). Microsatellite mapping of a gene affecting horn development in Bos taurus. Nature Genetics 4:206-210.
Grisart, B. et al. (2002) Genome Res. 12:222-231
Grosz, M D, Womack, J E, and Skow, L C (1992) Genomics, 14(4):863-868.
Harlizius B, Tammen I, Eichler K, Eggen A, Hetzel D J (1997). New markers on bovine chromosome 1 are closely linked to the polled gene in Simmental and Pinzgauer cattle. Mammalian Genome 8:255-257.
Hayes, B, and Goddard, M E, (2001) Genet. Sel. Evol. 33:209-229.
Hayes, B, Chamberlain, A. J., Goddard, M. E. (2006) Proc. 8^{th} WCGALP 22:(16).
Kaminski, S, Ahman, A, Rusc, A, Wojcik, E, and Malewski, T (2005) J. Appl. Genet. 46(1):45-58.
Kuhn, C. et al. (2004). Genetics 167:1873-81.
Kwok P Y, Methods for genotyping single nucleotide polymorphisms, (2001), Annu. Rev. Genomics Hum. Genet., 2:23 5-258.
Long C R, Gregory K E (1978). Inheritance of the horned scurred, and polled condition in cattle. Journal of Heredity 69:395-400.
Meuwissen, T H E, and Van Arendonk, J A M, (1992) J. Dairy Sci. 75:1651-1659.
Meuwissen, T H E, Hayes, B J, and Goddard, M E, (2001) Genetics. 157:1819-29.
Rothschild and Plastow, (1999), AgBioTechNet 10:1-8.
Schaeffer, L R (2006) J. Anim. Breed. Genet. 123:218-223.
Schmutz S M, Marquess F L S, Berryere T G, Moker J S (1995). DNA marker assisted selection of the polled condition in Charolais cattle. Mammalian Genome 6:710-713.
Schnabel, R. et al. (2005) PNAS 102:6896-6901.
Schrooten, C, Bovenhuis, H, van Arendonk, J A M, and Bijma, P (2005) J. Dairy Sci. 88:1569-1581.
Sharma, B S, Jansen, G B, Karrow, N A, Kelton, D, and Jiang, Z, (2006) J. Dairy Sci. 89:3653-3663.
Short, T H, et al. (1997) J. Anim. Sci. 75:3138-3142.
Spelman, R J and Bovenhuis, H, (1998) Animal Genetics, 29:77-84.
Spelman, R J and Garrick, D J, (1998) J. Dairy Sci, 81:2942-2950.
Stearns, T M, Beever, J E, Southey, B R, Ellis, M, McKeith, F K and Rodriguez-Zas, S L, (2005) J. Anim. Sci. 83:1481-93.
Syvänen A C, Accessing genetic variation: genotyping single nucleotide polymorphisms, (2001) Nat. Rev. Genet. 2:930-942.
VanRaden, P. M. and E. J. H. Klaaskate. 1993. J. Dairy Sci. 76:2758-2764.
Verrier, E, (2001) Genet. Sel. Evol. 33:17-38.
Villanueva, B, Pong-Wong, R, Fernandez, J, and Toro, M A (2005) J. Anim. Sci. 83:1747-52.
White W T, Ibsen H L (1936). Horn inheritance in Galloway-Holstein cattle crosses. Journal of Genetics 32:33-49.
Williams, J L, (2005), Rev. Sci. Tech. Off Int. Epiz. 24(1):379-391.
Windig, J J, and Meuwissen, T H E, (2004) J. Anim. Breed. Genet. 121:26-39.
Winter, A. et al. (2002). PNAS, 99:9300-9305.
Womack, J, (1987), Dev. Genet. 8(4):281-293.
Wunderlich K R, Abbey C A, Clayton D R, Song Y, Schein J E, Georges M, Coppieters W, Adelson D L, Taylor J F, Davis S L, Gill C A (2006). A 2.5-Mb contig constructed from Angus, Longhorn and horned Hereford DNA spanning the polled interval on bovine chromosome 1. Animal Genetics 37:592-594.
Yasue H, Kiuchi S, Hiraiwa H, Ozawa A, Hayashi T, (2006), Cytogenet. Genome Res., 112(1-4121-125.
Youngerman, S M, Saxton, A M, Oliver, S P, and Pighetti, G M, (2004) J. Dairy Sci. 87:2442-2448.

Patent Literature

| Patent/Publication | | | |
|---|---|---|---|
| Number | Title | Inventors | Pub. Date |
| U.S. Pat. No. | Genetic marker for superior milk | Cowan, Charles M.; | Aug. 20, 1991 |
| 5,041,371 | products in dairy cattle | Dentine, Margaret | |
| | | R.; Ax, Roy L.; | |
| | | Schuler, Linda A. | |
| U.S. Pat. No. | Allelic variants of bovine | Collier, Robert J.; | Dec. 20, 1994 |
| 5,374,523 | somatotropin gene: genetic marker | Hauser, Scott D.; | |
| | for superior milk production in | Krivi, Gwen G.; | |
| | bovine | Lucy, Matthew C. | |
| U.S. Pat. No. | Methods for testing bovine for | Lewin, Harris A.; | Dec. 10, 1996 |
| 5,582,987 | resistance or susceptibility to | van Eijk, Michiel J. | |
| | persistent lymphocytosis by | T. | |
| | detecting polymorphism in bola- | | |
| | dr3 exon 2 | | |
| U.S. Pat. No. | Genetic marker for improved milk | Tuggle, Christopher | Mar. 25, 1997 |
| 5,614,364 | production traits in cattle | K.; Freeman, Albert | |
| | | E. | |
| US2003039737A1 | Population of dairy cows | Cooper, Garth J. S. | Feb. 27, 2003 |
| | producing milk with desirable | | |
| | characteristics and methods of | | |
| | making and using same | | |
| US2004076977A1 | Marker assisted selection of | Georges, Michel | Apr. 22, 2004 |
| | bovine for improved milk | Alphonse Julien; | |
| | production using diacylglycerol | Coppieters, Wonter | |
| | acyltransferase gene dgat1 | Herman Robert; | |
| | | Grisart, Bernard | |
| | | Marie-Josee Jean; | |
| | | Shell, Russell Grant; | |
| | | Jean Reid, Suzanne; | |
| | | Ford, Christine Ann; | |
| | | Spelman, Richard | |
| | | John | |
| US2004234986A1 | Method of testing a mammal for | Fries, Hans-Rudolf; | Nov. 25, 2004 |
| | its predisposition for fat content | Winter, Andreas | |
| | of milk and/or its predisposition | | |
| | for meat marbling | | |
| US2004241723A1 | Systems and methods for | Marquess, Foley | Dec. 2, 2004 |
| | improving protein and milk | Leigh Shaw; | |
| | production of dairy herds | Laarveld, Bernard; | |
| | | Cleverly Buchanan, | |
| | | Fiona; Van Kessel, | |
| | | Andrew Gerald; | |
| | | Schmutz, Sheila | |
| | | Marie; Waldner, | |
| | | Cheryl; Christensen, | |
| | | David | |
| US2004254104A1 | Marker assisted selection of | Blott, Sarah; Kim, | Dec. 16, 2004 |
| | bovine for improved milk | Jong-Joo; Schmidt- | |
| | composition | Kuntzel, Anne; | |
| | | Cornet, Anne; Berzi, | |
| | | Paulette; Cambisano, | |
| | | Nadine; Grisart, | |
| | | Bernard; Karim, | |
| | | Latifa; Simon, | |
| | | Patricia; Georges, | |
| | | Michel; Farnir, | |
| | | Frederic; Coppieters, | |
| | | Wouter; Moisio, | |
| | | Sirja; Vilkki, | |
| | | Johanna; Spelman, | |
| | | Richard; Johnson, | |
| | | Dave; Ford, | |
| | | Christine; Snell, | |
| | | Russell | |
| US2005123929A1 | Methods and compositions for | Khatib, Hasan | Jun. 9, 2005 |
| | genetically detecting improved | | |
| | milk production traits in cattle | | |
| US2005136440A1 | Method for identifying animals | Renaville, Robert; | Jun. 23, 2005 |
| | for milk production qualities by | Gengler, Nicolas | |
| | analysing the polymorphism of | | |
| | the pit-1 and kappa-casein genes | | |
| US2005137805A1 | Gene expression profiles that | Lewin, Harris A.; | Jun. 23, 2005 |
| | identify genetically elite ungulate | Liu, Zonglin; | |
| | mammals | Rodriguez-Zas, | |
| | | Sandra; Everts, | |
| | | Robin E. | |
| US2005153317A1 | Methods and systems for inferring | DeNise, Sue; | Jul. 14, 2005 |
| | traits to breed and manage non- | Rosenfeld, David; | |
| | beef livestock | Kerr, Richard; | |
| | | Bates, Stephen; | |
| | | Holm, Tom | |
| US2005/053328A1 | Method and Markers for | DeNise et al | Jul. 14, 2005 |
| | Determining the Genotype of | | |
| | Horned/Polled Cattle | | |
| US2006037090A1 | Selecting animals for desired | Andersson, Leif; | Feb. 16, 2006 |
| | genotypic or potential phenotypic | Andersson, Goran; | |
| | properties | Georges, Michel; | |
| | | Buys, Nadine | |
| US2006094011A1 | Method for altering fatty acid | Morris, Christopher | May 4, 2006 |
| | composition of milk | Anthony; Tate, | |
| | | Michael Lewis | |
| US2006121472A1 | Method for determining the allelic | Prinzenberg, Eva- | Jun. 8, 2006 |
| | state of the 5′-end of the $g(a)s1- | Maria; Erhardt, | |
| | casein gene | George | |
| US2006166244A1 | Dna markers for increased milk | Schnabel, Robert D.; | Jul. 27, 2006 |
| | production in cattle | Sonstegard, Tad S.; | |
| | | Van Tassell, Curtis | |
| | | P.; Ashwell, Melissa | |
| | | S.; Taylor, Jeremy | |
| | | F. | |
| US2007/0134701 | Method and Markers for | DeNise et al | Jun. 14, 2007 |
| | Determining the Genotype of | | |
| | Horned/Polled Cattle | | |

### Description of the Tables

For each of the following tables, additional Information regarding the gene names and NCBI GeneID numbers can be found at www.ncbi.nlm.nih.gov/sites/entrez?db=Gene.

Table 1: provides the SEQ ID numbers of the SNPs associated with the polled/horned phenotype as described herein, the allele of each SNP as it corresponds to polled or horned, and the NCBI GeneID of the gene the SNP is located within.

Table 2: provides the SEQ ID NO of novel SNPs located between and/or in the 5′ or 3′ flanking DNA sequence of each of the SNPs listed in Table 1 and the gene the SNP is located within.

Table 3: provides a list of the genes containing SNPs associated with the polled/horned phenotype as described herein and the SEQ ID NO of all SNPs located within that gene as determined within the population analyzed.

TABLE 1
SNPs concordant with the polled trait.

| SEQ | Polled | Horned | | NCBI |
|---|---|---|---|---|
| ID | Allele | Allele | Gene | GeneID |
| 1 | C | T | IFNAR2 | 282258 |
| 2 | T | C | SYNJ1 | 282087 |
| 3 | T | C | SYNJ1 | 282087 |
| 4 | G | A | SYNJ1 | 282087 |
| 5 | T | C | SYNJ1 | 282087 |
| 6 | A | G | n.a. | n.a. |
| 7 | T | C | C21orf59 | 535482 |
| 8 | C | T | C21orf59 | 535482 |
| 9 | G | A | C21orf63 | 516536 |
| 10 | A | G | C21orf63 | 516536 |
| 11 | G | A | C21orf63 | 516536 |
| 12 | A | C | C21orf63 | 516536 |
| 13 | C | T | C21orf63 | 516536 |

n.a. means not applicable

TABLE 2
SNPs within the polled region on BTA01.

| SEQ | | NCBI |
|---|---|---|
| ID | Gene | GeneID |
| 14 | IFNAR2 | 282258 |
| 15 | IFNAR2 | 282258 |
| 16 | IFNAR2 | 282258 |
| 17 | IFNAR2 | 282258 |
| 18 | IFNAR2 | 282258 |
| 19 | IFNAR2 | 282258 |
| 20 | IFNAR2 | 282258 |
| 21 | IFNAR2 | 282258 |
| 22 | IFNAR2 | 282258 |
| 23 | IFNAR2 | 282258 |
| 24 | IFNAR2 | 282258 |
| 25 | IFNAR2 | 282258 |
| 26 | IFNAR2 | 282258 |
| 27 | IFNAR2 | 282258 |
| 28 | n.a. | n.a. |
| 29 | n.a. | n.a. |
| 30 | n.a. | n.a. |
| 31 | n.a. | n.a. |
| 32 | n.a. | n.a. |
| 33 | n.a. | n.a. |
| 34 | n.a. | n.a. |
| 35 | n.a. | n.a. |
| 36 | n.a. | n.a. |
| 37 | n.a. | n.a. |
| 38 | n.a. | n.a. |
| 39 | n.a. | n.a. |
| 40 | n.a. | n.a. |
| 41 | n.a. | n.a. |
| 42 | n.a. | n.a. |
| 43 | n.a. | n.a. |
| 44 | n.a. | n.a. |
| 45 | n.a. | n.a. |
| 46 | n.a. | n.a. |
| 47 | n.a. | n.a. |
| 48 | n.a. | n.a. |
| 49 | n.a. | n.a. |
| 50 | n.a. | n.a. |
| 51 | n.a. | n.a. |
| 52 | n.a. | n.a. |
| 53 | n.a. | n.a. |
| 54 | n.a. | n.a. |
| 55 | n.a. | n.a. |
| 56 | n.a. | n.a. |
| 57 | C21orf59 | 535482 |
| 58 | n.a. | n.a. |
| 59 | n.a. | n.a. |
| 60 | n.a. | n.a. |
| 61 | n.a. | n.a. |
| 62 | n.a. | n.a. |
| 63 | n.a. | n.a. |
| 64 | LOC784884 | 784884 |
| 65 | LOC784884 | 784884 |
| 66 | LOC784884 | 784884 |
| 67 | SYNJ1 | 282087 |
| 68 | SYNJ1 | 282087 |
| 69 | SYNJ1 | 282087 |
| 70 | SYNJ1 | 282087 |
| 71 | SYNJ1 | 282087 |
| 72 | SYNJ1 | 282087 |
| 73 | SYNJ1 | 282087 |
| 74 | SYNJ1 | 282087 |
| 75 | SYNJ1 | 282087 |
| 76 | SYNJ1 | 282087 |
| 77 | SYNJ1 | 282087 |
| 78 | SYNJ1 | 282087 |
| 79 | SYNJ1 | 282087 |
| 80 | SYNJ1 | 282087 |
| 81 | SYNJ1 | 282087 |
| 82 | SYNJ1 | 282087 |
| 83 | SYNJ1 | 282087 |
| 84 | SYNJ1 | 282087 |
| 85 | SYNJ1 | 282087 |
| 86 | C21orf63 | 516536 |
| 87 | C21orf63 | 516536 |
| 88 | C21orf63 | 516536 |
| 89 | C21orf63 | 516536 |
| 90 | C21orf63 | 516536 |
| 91 | C21orf63 | 516536 |
| 92 | C21orf63 | 516536 |
| 93 | C21orf63 | 516536 |
| 94 | C21orf63 | 516536 |
| 95 | C21orf63 | 516536 |
| 96 | C21orf63 | 516536 |
| 97 | C21orf63 | 516536 |
| 98 | C21orf63 | 516536 |
| 99 | C21orf63 | 516536 |
| 100 | C21orf63 | 516536 |
| 101 | C21orf63 | 516536 |
| 102 | C21orf63 | 516536 |
| 103 | C21orf63 | 516536 |
| 104 | C21orf63 | 516536 |
| 105 | C21orf63 | 516536 |
| 106 | C21orf63 | 516536 |
| 107 | C21orf63 | 516536 |
| 108 | C21orf63 | 516536 |
| 109 | C21orf63 | 516536 |
| 110 | C21orf63 | 516536 |
| 111 | C21orf63 | 516536 |
| 112 | C21orf63 | 516536 |
| 113 | C21orf63 | 516536 |
| 114 | C21orf63 | 516536 |
| 115 | C21orf63 | 516536 |
| 116 | C21orf63 | 516536 |
| 117 | C21orf63 | 516536 |
| 118 | C21orf63 | 516536 |
| 119 | C21orf63 | 516536 |
| 120 | C21orf63 | 516536 |
| 121 | C21orf63 | 516536 |
| 122 | C21orf63 | 516536 |
| 123 | C21orf63 | 516536 |
| 124 | C21orf63 | 516536 |
| 125 | C21orf63 | 516536 |
| 126 | C21orf63 | 516536 |
| 127 | C21orf63 | 516536 |
| 128 | C21orf63 | 516536 |
| 129 | C21orf63 | 516536 |
| 130 | C21orf63 | 516536 |
| 131 | C21orf63 | 516536 |
| 132 | C21orf63 | 516536 |
| 133 | C21orf63 | 516536 |
| 134 | C21orf63 | 516536 |
| 135 | C21orf63 | 516536 |
| 136 | C21orf63 | 516536 |
| 137 | C21orf63 | 516536 |
| 138 | n.a. | n.a. |
| 139 | n.a. | n.a. |
| 140 | n.a. | n.a. |
| 141 | n.a. | n.a. |
| 142 | n.a. | n.a. |
| 143 | n.a. | n.a. |
| 144 | n.a. | n.a. |
| 145 | n.a. | n.a. |

n.a. means not applicable

TABLE 3
Genes containing SNPs concordant with the
polled trait, and SNPs within those genes.

| | NCBI | SEQ |
|---|---|---|
| Gene | GeneID | ID |
| IFNAR2 | 282258 | 1 |
| IFNAR2 | 282258 | 14 |
| IFNAR2 | 282258 | 15 |
| IFNAR2 | 282258 | 16 |
| IFNAR2 | 282258 | 17 |
| IFNAR2 | 282258 | 18 |
| IFNAR2 | 282258 | 19 |
| IFNAR2 | 282258 | 20 |
| IFNAR2 | 282258 | 21 |
| IFNAR2 | 282258 | 22 |
| IFNAR2 | 282258 | 23 |
| IFNAR2 | 282258 | 24 |
| IFNAR2 | 282258 | 25 |
| IFNAR2 | 282258 | 26 |
| IFNAR2 | 282258 | 27 |
| SYNJ1 | 282087 | 2 |
| SYNJ1 | 282087 | 3 |
| SYNJ1 | 282087 | 4 |
| SYNJ1 | 282087 | 5 |
| SYNJ1 | 282087 | 67 |
| SYNJ1 | 282087 | 68 |
| SYNJ1 | 282087 | 69 |
| SYNJ1 | 282087 | 70 |
| SYNJ1 | 282087 | 71 |
| SYNJ1 | 282087 | 72 |
| SYNJ1 | 282087 | 73 |
| SYNJ1 | 282087 | 74 |
| SYNJ1 | 282087 | 75 |
| SYNJ1 | 282087 | 76 |
| SYNJ1 | 282087 | 77 |
| SYNJ1 | 282087 | 78 |
| SYNJ1 | 282087 | 79 |
| SYNJ1 | 282087 | 80 |
| SYNJ1 | 282087 | 81 |
| SYNJ1 | 282087 | 82 |
| SYNJ1 | 282087 | 83 |
| SYNJ1 | 282087 | 84 |
| SYNJ1 | 282087 | 85 |
| C21orf59 | 535482 | 7 |
| C21orf59 | 535482 | 8 |
| C21orf63 | 516536 | 9 |
| C21orf63 | 516536 | 10 |
| C21orf63 | 516536 | 11 |
| C21orf63 | 516536 | 12 |
| C21orf63 | 516536 | 13 |
| C21orf63 | 516536 | 86 |
| C21orf63 | 516536 | 87 |
| C21orf63 | 516536 | 88 |
| C21orf63 | 516536 | 89 |
| C21orf63 | 516536 | 90 |
| C21orf63 | 516536 | 91 |
| C21orf63 | 516536 | 92 |
| C21orf63 | 516536 | 93 |
| C21orf63 | 516536 | 94 |
| C21orf63 | 516536 | 95 |
| C21orf63 | 516536 | 96 |
| C21orf63 | 516536 | 97 |
| C21orf63 | 516536 | 98 |
| C21orf63 | 516536 | 99 |
| C21orf63 | 516536 | 100 |
| C21orf63 | 516536 | 101 |
| C21orf63 | 516536 | 102 |
| C21orf63 | 516536 | 103 |
| C21orf63 | 516536 | 104 |
| C21orf63 | 516536 | 105 |
| C21orf63 | 516536 | 106 |
| C21orf63 | 516536 | 107 |
| C21orf63 | 516536 | 108 |
| C21orf63 | 516536 | 109 |
| C21orf63 | 516536 | 110 |
| C21orf63 | 516536 | 111 |
| C21orf63 | 516536 | 112 |
| C21orf63 | 516536 | 113 |
| C21orf63 | 516536 | 114 |
| C21orf63 | 516536 | 115 |
| C21orf63 | 516536 | 116 |
| C21orf63 | 516536 | 117 |
| C21orf63 | 516536 | 118 |
| C21orf63 | 516536 | 119 |
| C21orf63 | 516536 | 120 |
| C21orf63 | 516536 | 121 |
| C21orf63 | 516536 | 122 |
| C21orf63 | 516536 | 123 |
| C21orf63 | 516536 | 124 |
| C21orf63 | 516536 | 125 |
| C21orf63 | 516536 | 126 |
| C21orf63 | 516536 | 127 |
| C21orf63 | 516536 | 128 |
| C21orf63 | 516536 | 129 |
| C21orf63 | 516536 | 130 |
| C21orf63 | 516536 | 131 |
| C21orf63 | 516536 | 132 |
| C21orf63 | 516536 | 133 |
| C21orf63 | 516536 | 134 |
| C21orf63 | 516536 | 135 |
| C21orf63 | 516536 | 136 |
| C21orf63 | 516536 | 137 |

What is claimed is:

1. A method for allocating an animal for use according to the animal's predicted horned/polled phenotype, the method comprising:
a. determining the animal's genotype at one or more locus/loci; wherein at least one locus contains a single nucleotide polymorphism (SNP), having at least two allelic variants; and wherein at least one SNP is selected from the SNPs described in Table 1;
b. analyzing the determined genotype of at least one evaluated animal at one or more SNPs selected from the SNPs described in Table 1 to determine which allelic variant is present;
c. correlating the identified allele with a horned or polled phenotype; and
d. allocating the animal for use according to its determined genotype.

2. The method of claim 1 wherein the animal's genotype is evaluated at two or more loci that contain SNPs selected from the SNPs described in Table 1.

3. The method of claim 1 wherein the animal's genotype is evaluated at two or more loci, including at least one that contains SNPs selected from the SNPs described in Table 1.

4. The method of claim 1 wherein the animal's genotype is evaluated at one or more SNPs selected from the SNPs located on Table 1 and wherein the animal's genotype is evaluated at one or more SNPs selected from the SNPs located on Table 2.

5. The method of claim 1 wherein the animal's genotype is evaluated at 10 or more SNPs, including at least one SNP selected from the SNPs described in Table 1.

6. The method of any of claims 3 to 5 wherein at least one evaluated SNP is associated with fitness.

7. The method of any of claims 3 to 5 wherein at least one evaluated SNP is associated with productivity.

8. The method of any of claims 1 to 7 that comprises whole-genome analysis.

9. A method for selecting a potential parent animal for breeding potential offspring comprising:
a. determining at least one potential parent animal's genotype at one or more genomic locus/loci; wherein at least one locus contains a single nucleotide polymorphism (SNP) that has at least two allelic variants, and wherein at least one SNP is selected from the SNPs described in Table 1;
b. analyzing the determined genotype of at least one evaluated animal for one or more SNPs selected from the SNPs described in Table 1 to determine which allele is present;
c. correlating the identified allele with a horned or polled phenotype;
d. allocating at least one animal for breeding use based on its genotype.

10. The method of claim 9 wherein the potential parent animal's genotype is evaluated at 1 or more loci that contain SNPs selected from the SNPs described in Table 1, and wherein the potential parent animal's genotype is evaluated at one or more loci that contain SNPs selected from the SNPs described in Table 2.

11. The method of claim 9 wherein the potential parent animal's genotype is evaluated at 2 or more loci that contain SNPs selected from the SNPs described in Table 1.

12. The method of claim 9 wherein the potential parent animal's genotype is evaluated at 10 or more loci, including at least one loci that contains a SNP selected from the SNPs described in Table 1.

13. The method of claim 9 wherein the potential parent animal's genotype is evaluated at 20 or more loci, including at least one loci that contain SNPs selected from the SNPs described in Table 1.

14. The method of any of claims 9 to 13 wherein the potential parent animal is selected to propagate the polled phenotype in the potential offspring.

15. The method of any of claims 9 to 13 wherein the potential parent animal is selected to propagate the horned phenotype in the potential offspring.

16. The method of any of claims 9 to 13 that comprises whole-genome analysis.

17. A method of producing progeny animals comprising:
a) identifying at least one potential parent animal that has been allocating for breeding in accordance with the method of claim 1;
b) producing progeny from the allocated animal through a process comprising:
i) natural breeding;
ii) artificial insemination;
iii) in vitro fertilization; and/or
iv) collecting semen/spermatozoa or at least one ovum from the animal and contacting it, respectively, with ovum/ova or semen/spermatozoa from a second animal to produce a conceptus by any means.

18. The method of claim 17 comprising producing progeny through natural breeding.

19. The method of claim 17 comprising producing offspring through artificial insemination, embryo transfer, and/or in vitro fertilization.

20. The method of claim 17 wherein the potential parent animal's genotype is evaluated at 1 or more loci that contain SNPs selected from the SNPs described in Table 1 and wherein the potential parent animal's genotype is evaluated at 1 or more loci that contain SNPs selected from the SNPs described in Table 2.

21. The method of claim 17 wherein the potential parent animal's genotype is evaluated at two or more loci that contain SNPs selected from the SNPs described in Table 1.

22. The method of claim 17 wherein the potential parent animal's genotype is evaluated at 10 or more loci, including at least one loci that contains a SNP selected from the SNPs described in Table 1.

23. The method of claim 17 wherein the potential parent animal's genotype is evaluated at 20 or more loci, including at least one loci that contain a SNP selected from the SNPs described in Table 1.

24. The method of any of claims 17 to 23 wherein the potential parent animal is selected to propagate the polled phenotype in the progeny.

25. The method of any of claims 17 to 23 wherein the potential parent animal is selected to propagate the horned phenotype in the progeny.

26. The method of any of claims 17 to 23 that comprises whole-genome analysis.

27. A nucleic acid array for determining which alleles are present in a sample; wherein the array comprises 2 or more nucleic acid sequences capable of hybridizing, under stringent conditions, with at least 1 or more SNPs selected from the group consisting of the SNPs described by Table 1.

28. The array of claim 27 wherein the array is capable of determining which allele is present for each of 2 or more SNPs selected from the group consisting of the SNPs described by Table 1.

29. The array of claim 27 wherein the array is capable of determining which allele is present for each of 10 or more SNPs, including at least one loci selected from the group consisting of the SNPs described by Table 1.

30. The array of claim 27 wherein the array is capable of determining which allele is present for each of 100 or more SNPs, including at least one loci selected from the group consisting of the SNPs described by Table 1.

31. A method of determining which alleles are present in an animal comprising:
a. Providing an array according to any of claims 27 to 30;
b. determining an animal's genotype using said array;
c. correlating at least one identified allele with a horned or polled phenotype; and
d. allocating at least one animal for breeding use based on its genotype.

32. The method of claim 31 wherein said animal is selected for the polled phenotype.

33. A method of identifying a genetic marker associated with the horned/polled phenotype by identifying a genetic marker in allelic association with at least one SNP selected from the group of SNPs described in Table 1, the method comprising:
a) identifying a genetic marker B₁ suspected of being in allelic association with a marker A₁ selected from the group of SNPs described in Table 1;
b) determining whether A₁ and B₁ are in allelic association;
wherein allelic association exists if r²>0.2 for Equation 1 for a population sample of at least 100 animals and wherein Equation 1 is:
$${r}^{2} = \frac{{[ f ( {A}_{1} {B}_{1} ) - f ( {A}_{1} ) f ( {B}_{1} ) ]}^{2}}{f ( {A}_{1} ) ( 1 - f ( {A}_{1} ) ) ( f ( {B}_{1} ) ( 1 - f ( {B}_{1} ) )} \qquad [ Equation 1 ]$$
and wherein A₁ represents an allele of a SNP described in Table 1; B₁ represents a genetic marker at another locus; f(A₁B₁) denotes frequency of having both A₁ and B₁; f(A₁) is the frequency of A₁ in the population; and f(B₁) is the frequency of B₁ in a population.

34. The method of claim 33 wherein the genetic marker B₁ is a SNP.

35. The method of claim 33 wherein the genetic marker identified is in linkage disequilibrium with at least one SNP selected from the group of SNPs described in Table 1.

36. The method of claim 33 wherein B₁ is a causal mutation underlying a quantitative or qualitative trait locus related to the horned/polled phenotype

37. The method of claim 33 wherein r²>0.5.

38. The method of claim 33 wherein r²>0.9.

39. The method of any of claims 33 to 38 for identifying a genetic marker in allelic association with a SNP associated with the polled phenotype.

40. The method of claim 39 wherein the genetic marker is in linkage disequilibrium with a SNP associated with the polled phenotype.

43. The method of claim 39 wherein the identified genetic marker is a causative mutation.

44. A method for allocating an animal for use according to the animal's predicted horned/polled phenotype, the method comprising:
a. determining the animal's genotype at one or more locus/loci; wherein at least one locus contains a genetic marker, having at least two allelic variants; and wherein at least one genetic marker is located in a gene described in Table 3;
b. analyzing the determined genotype of at least one evaluated animal at one or more SNPs loci within a gene described in table 3 to determine which allelic variant is present;
c. correlating the identified allele with a horned or polled phenotype; and
d allocating the animal for use according to its determined genotype.

45. The method of claim 44 wherein the animal's genotype is evaluated at two or more loci that contains a genetic marker located within a gene described in Table 3.

46. The method of claim 44 wherein the animal's genotype is evaluated at two or more loci, including at least one loci that contains a genetic marker located within a gene described in Table 3.

47. The method of claim 44 wherein said genetic marker is a polymorphism.

48. The method of claim 47 where said genetic marker is a single-nucleotide polymorphism (SNP).

49. The method of claim 44 wherein the animal's genotype is evaluated at two or more loci, at least one of which contains a genetic marker located within a gene described in Table 3.

50. The method of claim 49 wherein at least one evaluated marker is associated with fitness.

51. The method of claim 49 wherein at least one evaluated marker is associated with productivity.

52. The method of any of claims 44 to 51 that comprises whole-genome analysis.

53. A method for selecting a potential parent animal for breeding potential offspring:
a. determining at least one potential parent animal's genotype at one or more genomic locus/loci; wherein at least one loci contains a genetic markers located within a gene described in Table 3;
b. analyzing the determined genotype of at least one evaluated animal for one or more genetic markers to determine which allele is present;
c. correlating the identified allele with a horned or polled phenotype; and
d. allocating at least one animal for breeding use based on its genotype.

54. The method of claim 53 wherein said genetic marker is a polymorphism.

55. The method of claim 54 wherein said polymorphism is a single-nucleotide polymorphism (SNP).

56. The method of claim 53 wherein the potential parent animal's genotype is evaluated at two or more loci, at least one of which that contains a genetic marker located within a gene described in Table 3.

57. The method of 56 wherein the potential parent animal's genotype is evaluated at 10 or more loci, at least one of which that contains a genetic marker located within a gene described in Table 3.

58. The method of any of claims 53 to 56 wherein the potential parent animal is selected to propagate the polled phenotype in the potential offspring.

59. The method of any of claims 53 to 56 wherein the potential parent animal is selected to propagate the horned phenotype in the potential offspring.

60. The method of any of claims 53 to 56 that comprises whole-genome analysis.

61. A method of producing progeny animals comprising:
a. identifying at least one potential parent animal that has been allocating for breeding in accordance with the method of claim 53;
b. producing progeny from the allocated animal through a process comprising:
i) natural breeding;
ii) artificial insemination;
iii) in vitro fertilization; and/or
iv) collecting semen/spermatozoa or at least one ovum from the animal and contacting it, respectively, with ovum/ova or semen/spermatozoa from a second animal to produce a conceptus by any means.

62. The method of claim 61 comprising producing progeny through natural breeding.

63. The method of claim 61 comprising producing offspring through artificial insemination, embryo transfer, and/or in vitro fertilization.

64. The method of claim 61 wherein said genetic marker is a polymorphism.

65. The method of claim 61 where said genetic marker is a single-nucleotide polymorphism (SNP).

66. A method of identifying a genetic marker associated with the horned or polled phenotype by identifying a genetic marker in allelic association with at least one marker located in a gene described in Table 3, the method comprising:
a. identifying a genetic marker B₁ suspected of being in allelic association with a marker A₁ selected from the group of SNPs described in Table 3;
b. determining whether A₁ and B₁ are in allelic association;
wherein allelic association exists if r²>0.2 for Equation 1 for a population sample of at least 100 animals and wherein Equation 1 is:
$${r}^{2} = \frac{{[ f ( {A}_{1} {B}_{1} ) - f ( {A}_{1} ) f ( {B}_{1} ) ]}^{2}}{f ( {A}_{1} ) ( 1 - f ( {A}_{1} ) ) ( f ( {B}_{1} ) ( 1 - f ( {B}_{1} ) )} \qquad [ Equation 1 ]$$
and wherein A₁ represents an allele of a SNP described in Table 3; B₁ represents a genetic marker at another locus; f(A₁B₁) denotes frequency of having both A₁ and B₁; f(A₁) is the frequency of A₁ in the population; and f(B₁) is the frequency of B₁ in a population.

67. The method of claim 66 wherein the genetic marker B₁ is a SNP.

68. The method of claim 66 wherein the genetic marker identified is in linkage disequilibrium with at least one SNP located within a gene described in Table 3.

69. The method of claim 66 wherein B₁ is a causal mutation underlying a quantitative or qualitative trait locus related to the horned/polled phenotype.

70. The method of claim 66 wherein r²>0.5.

71. The method of claim 66 wherein r²>0.9.

72. The method of any of claims 66 to 71 for identifying a genetic marker in allelic association with a SNP associated with the polled phenotype.

73. The method of claim 72 wherein the genetic marker is in linkage disequilibrium with a SNP associated with the polled phenotype.

74. The method of claim 73 wherein the identified genetic marker is a causative mutation.

75. A method of selecting a bovine animal using gene expression comprising the following steps:
a. obtaining a sample from said animal;
b. analyzing said sample for gene expression; and
c. allocating said animal for use according to the analysis of gene expression;
Wherein said gene is selected from the genes described in Table 3.

76. The method of claim 75 wherein said gene expression is measured at the transcription level through analysis of mRNA.

77. The method of claim 75 wherein said gene expression is measured at the translational level through analysis of protein content of said sample.

78. The method of claim 77 wherein the protein content is analyzed using a test selected from the group consisting of western blot analysis, polyclonal antibody based tests, monoclonal antibody based tests, protein electrophoresis, protein assays, microarrays, immunohistochemistry, competition assays, enzyme assays, and an Enzyme-Linked ImmunoSorbent Assay (ELISA).

79. The method of claim 77 wherein the protein content is analyzed using a lateral flow test comprising antibodies.

80. The method of claim 77 wherein the protein content is analyzed using an Enzyme-Linked ImmunoSorbent Assay (ELISA).

81. A nucleic acid array for determining which allele of at least 10 polymorphisms are present in a sample; wherein the array comprises one or more nucleic acid sequences capable of hybridizing, under stringent conditions, with one or more polymorphisms selected from the polymorphisms described in Tables 1 and 2.

82. A nucleic acid array of claim 81 wherein the array comprises two or more polymorphisms selected from the polymorphisms described in Tables 1 and 2.

83. A nucleic acid array of claim 82 wherein the array comprises five or more polymorphisms selected from the polymorphisms described in Tables 1 and 2.

84. A nucleic acid array of claim 83 wherein the array comprises 10 or more polymorphisms selected from the polymorphisms described in Tables 1 and 2.

85. A method of evaluating an animal's genotype at one or more loci; wherein at least one locus comprises a polymorphism selected from the polymorphisms described in Table 1 and 2.

86. The method of claim 85 comprising evaluating an animal's genotype at 10 or more genomic locus/loci.

87. The method of claim 85 wherein the animal's genotype is evaluated at 10 or more loci, wherein at least two loci comprise a polymorphism selected from the polymorphisms described in Table 3 and the Sequence Listing.

88. The method of claim 85 wherein the animal's genotype is evaluated at 20 or more loci wherein at least two loci comprise a polymorphism selected from the polymorphisms described in Table 3 and the Sequence Listing.

89. The method of any of claims 85 through 88 that comprises whole-genome analysis.

90. An isolated nucleic acid sequence comprising at least 17 contiguous nucleotides of a nucleic acid selected from the group of nucleic acids described in Tables 1 & 2 and the Sequence Listing; wherein the nucleic acid comprises at least one polymorphic nucleotide described in Tables 1 and 2 and the Sequence Listing.

91. An isolated nucleic acid sequence comprising a sequence capable of hybridizing under stringent conditions to a nucleic acid sequence according to claim 90.


SEQUENCE LISTING (148 sequences)

SEQ ID NO: 1 - DNA, 143 bp, Bos taurus
  misc_feature (72)..(72): n is a, c, g, or t
ggctgcggggagcttggagaggggacgcccccgggcaggggtgcggtgtcgcgcgcggaaaaaccctgcganccggggcgcaggcggtaggaggagtcccgggcagataggctccaggagagggattccgggaaacggcggcg

SEQ ID NO: 2 - DNA, 143 bp, Bos taurus
  misc_feature (72)..(72): n is a, c, g, or t
tagcttgcaagaacacacaactgacaatagatttttctggtgagtttgtattacgttttcccttgtgatcangtgcagtgcagccactggtgagctggaacatggaaggcataacagttatttctagagtgggcaacggtggg

SEQ ID NO: 3 - DNA, 143 bp, Bos taurus
  misc_feature (72)..(72): n is a, c, g, or t
attggcagacagattcttcaccactggaccaccygggaagcccaggggactatttttaattactttttctanggtacgagagcatttgcactaattctacttgtatacatataaactctttatacatgcccacatgcttaact

SEQ ID NO: 4 - DNA, 143 bp, Bos taurus
  misc_feature (72)..(72): n is a, c, g, or t
gctgggagactgactcctgaaagccaaagcaaaacctcagaagtactgaaaggtagacccgtagtcaaaacngcagtgccattctgtatccttcccttccacttcttttcttttttctttcgcaatctctcactttatacaac

SEQ ID NO: 5 - DNA, 143 bp, Bos taurus
  misc_feature (72)..(72): n is a, c, g, or t
cttattgctttttgtttgttctgattattcagtgggctctctctttcccaggtgaaaacaaacggagtctcngccgtcagactggactcgccattaaagagtgacccatttgaagacttgtcattgaacctgcttgctgtatc

SEQ ID NO: 6 - DNA, 143 bp, Bos taurus
  misc_feature (72)..(72): n is a, c, g, or t
gtagtggagggaggcccgggtccccgcgcaccgtagcttccccagcctcagggtctgcccttgagccgtggnccccaacccccgccctcagatcgcgtttctctgttgctcctggcagcaggcacttctgagagcgggtactg

SEQ ID NO: 7 - DNA, 143 bp, Bos taurus
  misc_feature (72)..(72): n is a, c, g, or t
ctgagagcgggtactgtttccccctagtcccctccctctccccgctcccagatcgtgtttctctgttgctcntggcaacagccacttccgggagccgagccgtttcccccagttcccctgcccagtcccagcaccgcgaacgc

SEQ ID NO: 8 - DNA, 143 bp, Bos taurus
  misc_feature (72)..(72): n is a, c, g, or t
tttttagctccaaatgaaaaaatgaagcaagttataaagaagactatagaagaagccaaagcaataatatcnaaggttagctttttaaatgatattctaaaacttgtcttttgaaatacaaattccagttaatggcttccaga

SEQ ID NO: 9 - DNA, 143 bp, Bos taurus
  misc_feature (72)..(72): n is a, c, g, or t
ctgcctgccaggggctctctgaccctgcgagccttctgcggggggcgggggggagcaatgagtggaccaggntagasmcccagggattacgccaaggaccaaactgaggaggaggagagacgtcagggtgaagaggccagggc

SEQ ID NO: 10 - DNA, 143 bp, Bos taurus
  misc_feature (72)..(72): n is a, c, g, or t
ggatgctctaccctctagtgtcagctcacaaaccacaaacacatgcaaagcacatggtctgtcttgttgagnttcagtcactaagtcatgtccagctccctgcaaccccaygaactgcagcatgtcaggcttccctgtccttc

SEQ ID NO: 11 - DNA, 143 bp, Bos taurus
  misc_feature (72)..(72): n is a, c, g, or t
ttaccttcagacagtyccaaacttgggtggtaacctatagactgcttcaaggattgcaaccgcttcactaangaaacaagctctaagtgcctgtaatctgtgagaacaatctaatggttacaccgcaacggtacctaaagcaa

SEQ ID NO: 12 - DNA, 143 bp, Bos taurus
  misc_feature (72)..(72): n is a, c, g, or t
taaagcaatatttgttcagttacagtgacgtcacactgtagaaaggcttgttggctttctgttctttgctcnacaatattctcagcctgcyggtggctgaattcagattgctttccatagcttaaatcagcaaagacagaact

SEQ ID NO: 13 - DNA, 143 bp, Bos taurus
  misc_feature (72)..(72): n is a, c, g, or t
ttacagtgacgtcacactgtagaaaggcttgttggctttctgttctttgctcmacaatattctcagcctgcnggtggctgaattcagattgctttccatagcttaaatcagcaaagacagaacttttctagcagycaagaaag

SEQ ID NO: 14 - DNA, 143 bp, Bos taurus
  misc_feature (72)..(72): n is a, c, g, or t
cagtcataaattgagcctggagagagtattgtttgcttttctaacatgtttttcttctttccagtgcacatnagcctcgtgtttggtatttcgtatgttgcgcctggtaagagattytcttgacttaacaaaattttgtctaa

SEQ ID NO: 15 - DNA, 143 bp, Bos taurus
  misc_feature (72)..(72): n is a, c, g, or t
atgtttttcttctttccagtgcacatmagcctcgtgtttggtatttcgtatgttgcgcctggtaagagattntcttgacttaacaaaattttgtctaagggtgtgagtgggggagcaaccatttagaagtcaaaaaatgtggg

SEQ ID NO: 16 - DNA, 143 bp, Bos taurus
  misc_feature (72)..(72): n is a, c, g, or t
agaagtcaaaaaatgtgggaaattggatttctgagtccaaatctgttgccattttgtttgaatctttttccncaaatactttctctgtagaaggagaatttagccaattctgattttcacatttgtctcataaaactattaca

SEQ ID NO: 17 - DNA, 143 bp, Bos taurus
  misc_feature (72)..(72): n is a, c, g, or t
catcacatacaggctaacactttctttgaccatctctttgcctttcaaagtaaacgagaagatatgaaggtngtcaagractgtataaacgtcacaagatcgttttgtgacctcacagacgtgtgggtaaacacgacggacat

SEQ ID NO: 18 - DNA, 143 bp, Bos taurus
  misc_feature (72)..(72): n is a, c, g, or t
tacaggctaacactttctttgaccatctctttgcctttcaaagtaaacgagaagatatgaaggtsgtcaagnactgtataaacgtcacaagatcgttttgtgacctcacagacgtgtgggtaaacacgacggacatgtacatc

SEQ ID NO: 19 - DNA, 143 bp, Bos taurus
  misc_feature (72)..(72): n is a, c, g, or t
gtacatcccccaggtggttggattcagagagaatgcaaagctggtcatttgtatgggctctttcttcctggngccagacagtgagtttgtcttgtttattaccttcatcatcatcgctaagcacatcgttacttgctttgcca

SEQ ID NO: 20 - DNA, 143 bp, Bos taurus
  misc_feature (72)..(72): n is a, c, g, or t
ctcagtttttcaccaaataaaatatgcatatatataacccatgcctcgagggccacaggaaatcaaaaaganagctgtaaagaggtatggttgtatcaggacaatggttatcattacggttatgctatttattgattaacctt

SEQ ID NO: 21 - DNA, 143 bp, Bos taurus
  misc_feature (72)..(72): n is a, c, g, or t
gaggacagtgactccacggaggggtctgagggcagaatcgtcttcaacgtgaatttgaactctgtgtgtgtnagagcccttgaggatgacaaggactcagaagtcactctaatgtccccatcttgtccagaagagacagtcga

SEQ ID NO: 22 - DNA, 143 bp, Bos taurus
  misc_feature (72)..(72): n is a, c, g, or t
aacttggacacacgctgcctctggacatcaccccccacccccagcacctgcacttgctcctttggggcctcngagtgttctccctggggaggagtaggacactggtgacatcatctatgcaaatgctcactctgkggggaggg

SEQ ID NO: 23 - DNA, 143 bp, Bos taurus
  misc_feature (72)..(72): n is a, c, g, or t
ggggcctcygagtgttctccctggggaggagtaggacactggtgacatcatctatgcaaatgctcactctgnggggaggggaggtgggaaacagagcattctgctattattcagtgcactgtttacctgttcaaggcaatttg

SEQ ID NO: 24 - DNA, 143 bp, Bos taurus
  misc_feature (72)..(72): n is a, c, g, or t
ggaggtgggaaacagagcattctgctattattcagtgcactgtttacctgttcaaggcaatttgccttgatngggaaaatgccctgcctgttccctttcctgtcatggatgtctaatgattctgcatcatgtttyccagagta

SEQ ID NO: 25 - DNA, 143 bp, Bos taurus
  misc_feature (72)..(72): n is a, c, g, or t
gccttgatrgggaaaatgccctgcctgttccctttcctgtcatggatgtctaatgattctgcatcatgtttnccagagtaaagaacaaagttccctgtggttttcaggcaatgttccagggccctgtaagatttctgggaaat

SEQ ID NO: 26 - DNA, 143 bp, Bos taurus
  misc_feature (72)..(72): n is a, c, g, or t
ctgggttacacaaaactccctctacttcgtggcctctgggagccaaagggaccttctgcctccctgagctcntgttcctgtggaatgttctagcgacagaagtcagccagtgtcttgcatcacctagtacttaccctgctctg

SEQ ID NO: 27 - DNA, 143 bp, Bos taurus
  misc_feature (72)..(72): n is a, c, g, or t
tctggggcacccctagataatattgtgtcgcctacactcttgagaattcaactctcagccctcctcaatctnagtgaggaagacagacacacacacacataataactctggtgccagacaggtgactaatagagaaacatttg

SEQ ID NO: 28 - DNA, 143 bp, Bos taurus
  misc_feature (72)..(72): n is a, c, g, or t
taggcaaactaacacatcttcccccctgcttttaacaggaatgtctagcttcaagctgcctgtgctagaggnaggcatagccaaggagaagctgaatggggccagmgcagtcagttcaccccaggccagaggccacygggaga

SEQ ID NO: 29 - DNA, 143 bp, Bos taurus
  misc_feature (72)..(72): n is a, c, g, or t
acaggaatgtctagcttcaagctgcctgtgctagaggyaggcatagccaaggagaagctgaatggggccagngcagtcagttcaccccaggccagaggccacygggagaccctgctgcgttgactcattacagcgaaacgtgc

SEQ ID NO: 30 - DNA, 143 bp, Bos taurus
  misc_feature (72)..(72): n is a, c, g, or t
tagaggyaggcatagccaaggagaagctgaatggggccagmgcagtcagttcaccccaggccagaggccacngggagaccctgctgcgttgactcattacagcgaaacgtgcagccaaagaaactgaaaaacttcacgtgttg

SEQ ID NO: 31 - DNA, 143 bp, Bos taurus
  misc_feature (72)..(72): n is a, c, g, or t
ccagmgcagtcagttcaccccaggccagaggccacygggagaccctgctgcgttgactcattacagcgaaangtgcagccaaagaaactgaaaaacttcacgtgttggcccaacccctgaaaatggtcggaaaaggagactcc

SEQ ID NO: 32 - DNA, 143 bp, Bos taurus
  misc_feature (72)..(72): n is a, c, g, or t
cccaacccctgaaaatggtcggaaaaggagactccaggaaacaaatggacttatgcttcatcatcagaaagntctgctgtgtttacagacagaagccagcatgaaaacaataggaacaaaaatagctgcgcatgtcagctggg

SEQ ID NO: 33 - DNA, 143 bp, Bos taurus
  misc_feature (72)..(72): n is a, c, g, or t
cccgcaccgtgatccccgccgcccacgccggcccgcgttaccttggcagtgtggcctcgccgctgccctccnggtgccagcctcgggcccacccgcttcagccggactccgccgccgcccctccgcaccccggctcggcgctc

SEQ ID NO: 34 - DNA, 143 bp, Bos taurus
  misc_feature (72)..(72): n is a, c, g, or t
cccagaggaaacccacaggccacttcgtgtgagggggtgattaaaaacaaaaatggggagtttacagcttangttaaaattagcctaggcgaatgtgatgaaggaccaccccccgcccccgccaccaactgtcctggtcagct

SEQ ID NO: 35 - DNA, 143 bp, Bos taurus
  misc_feature (72)..(72): n is a, c, g, or t
ttttgggctgtgttgggtcttagttgcagcacatgggatctttgttgtggcaggcgggcttagctgccctgngggatccagttccccgaccaggaatggaacccttgtcccctgccttggaaggtggattcttaaccactgaa

SEQ ID NO: 36 - DNA, 143 bp, Bos taurus
  misc_feature (72)..(72): n is a, c, g, or t
ggatccagttccccgaccaggaatggaacccttgtcccctgccttggaaggtggattcttaaccactgaacnaccagggaagttccctagctttccatcttttataamaattaagctctacctccaatgtcctgatgtgccca

SEQ ID NO: 37 - DNA, 143 bp, Bos taurus
  misc_feature (72)..(72): n is a, c, g, or t
ccctgccttggaaggtggattcttaaccactgaacyaccagggaagttccctagctttccatcttttataanaattaagctctacctccaatgtcctgatgtgcccagtaagtacaagtaaccttatggtamaccaagggcac

SEQ ID NO: 38 - DNA, 143 bp, Bos taurus
  misc_feature (72)..(72): n is a, c, g, or t
atcttttataamaattaagctctacctccaatgtcctgatgtgcccagtaagtacaagtaaccttatggtanaccaagggcacttttagcaaagaatcctaagatcccctggagaaggaaatggcaacccactytagtattct

SEQ ID NO: 39 - DNA, 143 bp, Bos taurus
  misc_feature (72)..(72): n is a, c, g, or t
cttatggtamaccaagggcacttttagcaaagaatcctaagatcccctggagaaggaaatggcaacccactntagtattcttgtctgaaaaatcytgtggacagaggagcctggwgggctatagtccatggggtcacaaagag

SEQ ID NO: 40 - DNA, 143 bp, Bos taurus
  misc_feature (72)..(72): n is a, c, g, or t
ttagcaaagaatcctaagatcccctggagaaggaaatggcaacccactytagtattcttgtctgaaaaatcntgtggacagaggagcctggwgggctatagtccatggggtcacaaagaggcaaagctgagcagcacccctgc

SEQ ID NO: 41 - DNA, 143 bp, Bos taurus
  misc_feature (72)..(72): n is a, c, g, or t
cccctggagaaggaaatggcaacccactytagtattcttgtctgaaaaatcytgtggacagaggagcctggngggctatagtccatggggtcacaaagaggcaaagctgagcagcacccctgcttttagcagaggattgttct

SEQ ID NO: 42 - DNA, 143 bp, Bos taurus
  misc_feature (72)..(72): n is a, c, g, or t
gagcagagaggggagggaggtgaaggtgggcacgtgtctgggaggggctgggaaaaccctctgtggccctcntctaaagtcactacgtgggtcactgtgtgggagggttgctagatttagcaaataaaaagacaagatgccct

SEQ ID NO: 43 - DNA, 143 bp, Bos taurus
  misc_feature (72)..(72): n is a, c, g, or t
agatctgtaataaatttttggcataatatactaactttttataatatatgtatatataatttaatacatgcnttagtacatctttgtcctaagttacagtatatatatgtattttagtacatcccatgccatatttgggaaat

SEQ ID NO: 44 - DNA, 143 bp, Bos taurus
  misc_feature (72)..(72): n is a, c, g, or t
gttacagtatatatatgtattttagtacatcccatgccatatttgggaaatattcatactaaaaaaatttcnccatttatctgaaatatattatatataatatttcataatatataatatatatttatttgaaatgtagcata

SEQ ID NO: 45 - DNA, 143 bp, Bos taurus
  misc_feature (72)..(72): n is a, c, g, or t
atttatctgaaatatattatatataatatttcataatatataatatatatttatttgaaatgtagcatatangcatcaaattgggctagatgtcctgtattgtatctgtgaacagtggccccagagcagrccatatgcataat

SEQ ID NO: 46 - DNA, 143 bp, Bos taurus
  misc_feature (72)..(72): n is a, c, g, or t
aatgtagcatataygcatcaaattgggctagatgtcctgtattgtatctgtgaacagtggccccagagcagnccatatgcataataaataatacatatttatttgaaatgtaayatatacccatcaaattggactgggtgtcc

SEQ ID NO: 47 - DNA, 143 bp, Bos taurus
  misc_feature (72)..(72): n is a, c, g, or t
tgtatctgtgaacagtggccccagagcagrccatatgcataataaataatacatatttatttgaaatgtaanatatacccatcaaattggactgggtgtcctgtattttagctgtgagcagtgggcccagagcaggccatggc

SEQ ID NO: 48 - DNA, 143 bp, Bos taurus
  misc_feature (72)..(72): n is a, c, g, or t
tttgaggagataatgtgaaatgtaggagtatgtagattgtgcttactgagttcaagaggttggatgggaagngaagcraagtgaaagttgctcagtcatgtccaactstttgtgaccttatggactatgcagttcatgkaatt

SEQ ID NO: 49 - DNA, 143 bp, Bos taurus
  misc_feature (72)..(72): n is a, c, g, or t
gagataatgtgaaatgtaggagtatgtagattgtgcttactgagttcaagaggttggatgggaagygaagcnaagtgaaagttgctcagtcatgtccaactstttgtgaccttatggactatgcagttcatgkaattctccag

SEQ ID NO: 50 - DNA, 143 bp, Bos taurus
  misc_feature (72)..(72): n is a, c, g, or t
ttgtgcttactgagttcaagaggttggatgggaagygaagcraagtgaaagttgctcagtcatgtccaactntttgtgaccttatggactatgcagttcatgkaattctccaggccagaatactggagtgggtagcctttccc

SEQ ID NO: 51 - DNA, 143 bp, Bos taurus
  misc_feature (72)..(72): n is a, c, g, or t
gaagygaagcraagtgaaagttgctcagtcatgtccaactstttgtgaccttatggactatgcagttcatgnaattctccaggccagaatactggagtgggtagcctttcccttctccaagggatcttcctagcccagggata

SEQ ID NO: 52 - DNA, 143 bp, Bos taurus
  misc_feature (72)..(72): n is a, c, g, or t
agcttgaaggactttgagcattatcttgctggggtgtgaaatgattacaactgtatggtagtttgagcattngttggcattgcccttctttgggattggaatgaaaactgaccttttccagtcgtatggccactgctgagttt

SEQ ID NO: 53 - DNA, 143 bp, Bos taurus
  misc_feature (72)..(72): n is a, c, g, or t
ggaatgaaaactgaccttttccagtcgtatggccactgctgagttttccaaatttgctgacatattgagtgnagcactttaagagcatcatcttttaggattttaaatagctcagctggaattccatcacctccactagcttt

SEQ ID NO: 54 - DNA, 144 bp, Bos taurus
  misc_feature (72)..(73): n is a, c, g, or t
gctggtttctttgccaagtcctccctagccaggccctctcctggtcctgacacctgtagatccctgtcagcnnctggagggctccagaccactgcggacaagctccgggctgtgactgctccaggctgaaaactgcagcctact

SEQ ID NO: 55 - DNA, 143 bp, Bos taurus
  misc_feature (72)..(72): n is a, c, g, or t
cctccaattccagcccggagtaaatcccaggaaaacacgcgatgttccccaaacccattcatcccaagctcnagcagcacaaatcctttcaccgacaggaccgccgctcctggaaacccctttcgagctgagtctcaagaatc

SEQ ID NO: 56 - DNA, 143 bp, Bos taurus
  misc_feature (72)..(72): n is a, c, g, or t
tcattctttctttttctctctgtttctctttctgtctgtctctctcctcctctcccaccgcaccggcccccngccccctgacccttccttccagaggcaaaggaaacaaacatgtttagagtgggccttagcttgtttttttc

SEQ ID NO: 57 - DNA, 143 bp, Bos taurus
  misc_feature (72)..(72): n is a, c, g, or t
agacgtaccaagaagctttcagactacgtggggaagaatgaaaaaaccaaaatcatcgtcaagattcagcangtaagcgctttgactttaatgtgtcacggctggttttttttaacttactttcacactggtaaaaatttaca

SEQ ID NO: 58 - DNA, 143 bp, Bos taurus
  misc_feature (72)..(72): n is a, c, g, or t
ctgtgggcctgggcccctcctggggaggcccgatacatgtgatgggggtttctgacttggaaacaatttcanaagccatcctgggagtaggtaaggcaagagrggcccccagaacctcaggcccgatcctcccgggtttctga

SEQ ID NO: 59 - DNA, 143 bp, Bos taurus
  misc_feature (72)..(72): n is a, c, g, or t
gatacatgtgatgggggtttctgacttggaaacaatttcawaagccatcctgggagtaggtaaggcaagagnggcccccagaacctcaggcccgatcctcccgggtttctgagaacctggccgctgttttctttcccccaaca

SEQ ID NO: 60 - DNA, 143 bp, Bos taurus
  misc_feature (72)..(72): n is a, c, g, or t
cgttggtctgggcttggtgcccatccaaacattctcagcaggactcctcagcctccttccttgagaagaccntggcattcttctcgtgggctcggaaaatgcaaaygaacaacttactgcatttktgcaagccaaagactgct

SEQ ID NO: 61 - DNA, 143 bp, Bos taurus
  misc_feature (72)..(72): n is a, c, g, or t
tcagcaggactcctcagcctccttccttgagaagaccrtggcattcttctcgtgggctcggaaaatgcaaangaacaacttactgcatttktgcaagccaaagactgctcttcgccttggctggcctgtttcccagctgcctc

SEQ ID NO: 62 - DNA, 143 bp, Bos taurus
  misc_feature (72)..(72): n is a, c, g, or t
tccttccttgagaagaccrtggcattcttctcgtgggctcggaaaatgcaaaygaacaacttactgcatttntgcaagccaaagactgctcttcgccttggctggcctgtttcccagctgcctctctgggggtcctgggagga

SEQ ID NO: 63 - DNA, 143 bp, Bos taurus
  misc_feature (72)..(72): n is a, c, g, or t
tgcaagccaaagactgctcttcgccttggctggcctgtttcccagctgcctctctgggggtcctgggaggangtgcatgcgtgtggaggttggggggagacacaggtaagagaagaaccattgtggcattcaacaagagcttt

SEQ ID NO: 64 - DNA, 143 bp, Bos taurus
  misc_feature (72)..(72): n is a, c, g, or t
gtccccaggcctaaccccgagcccgttaggccggtgcgggaggcggggggtggtcctgacaccctattgggncaagcccccgaagtttttcctcagagtccctccccggcaccccaggggagggtcggtaagctctgaggaga

SEQ ID NO: 65 - DNA, 143 bp, Bos taurus
  misc_feature (72)..(72): n is a, c, g, or t
cccccttagcggggaaggggcggggtgtcgtgggaggtcccgccccactccctgactgcccccgcgggtcgnttaaccgtgagctgcccgccgcctccgcgcgcagcgaccttcgcttcgggcggcgtgtttccccaggcggc

SEQ ID NO: 66 - DNA, 143 bp, Bos taurus
  misc_feature (72)..(72): n is a, c, g, or t
cgctgacgggtccgggggccggtaaatatcaatggccacccggggtggggagaagagtgacgactcgggacnggaccggctcctcggggggtcccgggccgtcgacagcccggaaagcggctccttctcaggccaggacgcaa

SEQ ID NO: 67 - DNA, 143 bp, Bos taurus
  misc_feature (72)..(72): n is a, c, g, or t
aaataaccacactgtcagattttaaaagacagagacactagaagaaagttggaagaaattttgaatagcttntagaatattggcacttcccatcccattaagtctttggtttttgcaggtgatattatgttacattatctggt

SEQ ID NO: 68 - DNA, 143 bp, Bos taurus
  misc_feature (72)..(72): n is a, c, g, or t
agtggggagaaagtagatgctacattttaatcagcttaatttttctcagtttgttttagcttttgttatttnggttttaaattgcccatttagtttcactgaatttgtttcaggtgccagagtggtacagttcgaacaaactg

SEQ ID NO: 69 - DNA, 143 bp, Bos taurus
  misc_feature (72)..(72): n is a, c, g, or t
tttactgctctggatgtcatcagtggatgcagctgtggcactgagccccttctgtctccctgcagtgctgtnatccaaaagaggaaaggaaacagtgacaccttaatgtgacattttaatgtgacagtttgatattcagaatg

SEQ ID NO: 70 - DNA, 143 bp, Bos taurus
  misc_feature (72)..(72): n is a, c, g, or t
ccttttactacagtgaagtcgtatacaacttatctaaagtttcagaatacctattgagtgaaaacaatgtanaatgaaagtccatgcttttttccccaacaggctggaaagttaaaagatggtgctcgttcagttagtagaac

SEQ ID NO: 71 - DNA, 143 bp, Bos taurus
  misc_feature (72)..(72): n is a, c, g, or t
atgttttgctcctgggaaatactctaaatagtgatttagctgacaaagctcgagcccttttaactactggangtttgcgtggtaggcgtactttatattttactttatgttgtctgtattccttctaaaagtccaagaggtga

SEQ ID NO: 72 - DNA, 143 bp, Bos taurus
  misc_feature (72)..(72): n is a, c, g, or t
ggtaggcgtactttatattttactttatgttgtctgtattccttctaaaagtccaagaggtgaatgtaaacntatttacatatatactttctagtttgaattgattaaaatagttttttgaaagaatttctatcattgtaatt

SEQ ID NO: 73 - DNA, 143 bp, Bos taurus
  misc_feature (72)..(72): n is a, c, g, or t
cataaaggtgtaattgataattgtcctacagaaataggctgtattactgtcaccgtctttttgaagattccnttgttaatattttctggtagcacaacaaatcagaagctctgggctgcggaacttcagaagaccatctccag

SEQ ID NO: 74 - DNA, 143 bp, Bos taurus
  misc_feature (72)..(72): n is a, c, g, or t
tataaagaagtaattgcagttcagggtccaccagatggtacggtgttggtctcaatcaaaagctctttaccngaaaataattttttcaacgatgctttgattgatgagcttttacagcagtttacaaatttcggtgaagttat

SEQ ID NO: 75 - DNA, 143 bp, Bos taurus
  misc_feature (72)..(72): n is a, c, g, or t
atcttcccagaccagggattgaacccatgtccatctgcattggcagacagattcttcaccactggaccaccngggaagcccaggggactatttttaattactttttctayggtacgagagcatttgcactaattctacttgta

SEQ ID NO: 76 - DNA, 143 bp, Bos taurus
  misc_feature (72)..(72): n is a, c, g, or t
tatgacttcccgggctggtatgtttgatttagatggttaaacctccgatgccttcagatgtcctttggaganctatttttgcttaactttcacatccataggctaaagtgtagcatgttttctgtgcgtacaaaagaggaaac

SEQ ID NO: 77 - DNA, 143 bp, Bos taurus
  misc_feature (72)..(72): n is a, c, g, or t
actccgtgcgaccccatagacggcagcccaccaggctcccccatccctgggattctccaggcaagaacactngagtgggttgccatttccttctccaatgcatgaaagtgaaaagtgaaagtgaagtcgctcagtcgtgtccg

SEQ ID NO: 78 - DNA, 143 bp, Bos taurus
  misc_feature (72)..(72): n is a, c, g, or t
tccttctccaatgcatgaaagtgaaaagtgaaagtgaagtcgctcagtcgtgtccgactcttagcgaccccntggactgcagcctacagstcctctgtccatgggattttccaggcaagactactggagtggggtgccattgc

SEQ ID NO: 79 - DNA, 143 bp, Bos taurus
  misc_feature (72)..(72): n is a, c, g, or t
aagtgaaaagtgaaagtgaagtcgctcagtcgtgtccgactcttagcgaccccrtggactgcagcctacagntcctctgtccatgggattttccaggcaagactactggagtggggtgccattgccttctccgactatatagc

SEQ ID NO: 80 - DNA, 143 bp, Bos taurus
  misc_feature (72)..(72): n is a, c, g, or t
gtacatataactgaatcactttgctgtacagcagaaattaataacattgtagatcaacttaataaagtaaantttaaaaagatatttaggaaaatgtaccttattccccatgcttgataattcaaaatacatgataaaggata

SEQ ID NO: 81 - DNA, 143 bp, Bos taurus
  misc_feature (72)..(72): n is a, c, g, or t
ttgcgtcctggcctgagaaggagccgctttccgggctgtcgacggcccgggaccccccgaggagccggtccngtcccgagtcgtcactcttctccccaccccgggtggccattgatatttaccggcccccggacccgtcagcg

SEQ ID NO: 82 - DNA, 143 bp, Bos taurus
  misc_feature (72)..(72): n is a, c, g, or t
acttgtcccatttactgttggtgttcactctgataacttgtgttgatgtggtatctgccaggcgtcactggncagctcttttttccttataattaataagtgtttgaaggtgaggtactttgagactgtgtatgatcccattg

SEQ ID NO: 83 - DNA, 143 bp, Bos taurus
  misc_feature (72)..(72): n is a, c, g, or t
atttgctaagataagaaatagggtaggtcatgagtaggtttacaaaactcattttgacatttattgtccctntaacagaacartactgattcatccttgtaaaaactcatcactgagtttgggtactctaccaccaagctctt

SEQ ID NO: 84 - DNA, 143 bp, Bos taurus
  misc_feature (72)..(72): n is a, c, g, or t
taagaaatagggtaggtcatgagtaggtttacaaaactcattttgacatttattgtccctktaacagaacantactgattcatccttgtaaaaactcatcactgagtttgggtactctaccaccaagctcttaatagccagta

SEQ ID NO: 85 - DNA, 143 bp, Bos taurus
  misc_feature (72)..(72): n is a, c, g, or t
cacgctcattgtctctctctgtctcccacgcttgctcgctctcttttctcatcccgcttttgggtcgggtcngcatgccctcatgcctcgaggttgtatcttcctttattttctaaataaaactgtgggagctgtaacactgt

SEQ ID NO: 86 - DNA, 143 bp, Bos taurus
  misc_feature (72)..(72): n is a, c, g, or t
ctgggtcaggaagatcctctggagaagggatagctacccactccagtattcttgggcttccttggtggctcngctggtaaaggatccgcctgccatgtgggagacctgggttacatccctgggttgggatgtaacattacatt

SEQ ID NO: 87 - DNA, 143 bp, Bos taurus
  misc_feature (72)..(72): n is a, c, g, or t
tgtcacatggtctctggccaaggcagatcacaggagggggctacctgaccccagtccagccaaagaaacagntctctggtaggaatttggaaggaagttgccagattggtagtctttataaattgttaggacagaaaagtgta

SEQ ID NO: 88 - DNA, 143 bp, Bos taurus
  misc_feature (72)..(72): n is a, c, g, or t
tctggtaggaatttggaaggaagttgccagattggtagtctttataaattgttaggacagaaaagtgtaacntaagaatcgcctgggttgtggggatgactgtatgccatctagcccargctcagagaaaagccgtagagggg

SEQ ID NO: 89 - DNA, 143 bp, Bos taurus
  misc_feature (72)..(72): n is a, c, g, or t
attgttaggacagaaaagtgtaacktaagaatcgcctgggttgtggggatgactgtatgccatctagcccangctcagagaaaagccgtagaggggagagagaggagcagagagacagacagacagagagactccatgctcgc

SEQ ID NO: 90 - DNA, 143 bp, Bos taurus
  misc_feature (72)..(72): n is a, c, g, or t
agggtgtgttgggcagatgccatgacttgttgctccaaacctccttctgttcacacgaccaccgcctccccnggaggcaaaatctccaaamggctggcrgagggtgtatccttaccacaygcgtagaagacygagagatattt

SEQ ID NO: 91 - DNA, 143 bp, Bos taurus
  misc_feature (72)..(72): n is a, c, g, or t
ccatgacttgttgctccaaacctccttctgttcacacgaccaccgcctccccmggaggcaaaatctccaaanggctggcrgagggtgtatccttaccacaygcgtagaagacygagagatattttttcactcctggcagacag

SEQ ID NO: 92 - DNA, 143 bp, Bos taurus
  misc_feature (72)..(72): n is a, c, g, or t
tgttgctccaaacctccttctgttcacacgaccaccgcctccccmggaggcaaaatctccaaamggctggcngagggtgtatccttaccacaygcgtagaagacygagagatattttttcactcctggcagacaggggcttcc

SEQ ID NO: 93 - DNA, 143 bp, Bos taurus
  misc_feature (72)..(72): n is a, c, g, or t
gttcacacgaccaccgcctccccmggaggcaaaatctccaaamggctggcrgagggtgtatccttaccacangcgtagaagacygagagatattttttcactcctggcagacaggggcttccgaaatggtggttgttgacgag

SEQ ID NO: 94 - DNA, 143 bp, Bos taurus
  misc_feature (72)..(72): n is a, c, g, or t
accgcctccccmggaggcaaaatctccaaamggctggcrgagggtgtatccttaccacaygcgtagaagacngagagatattttttcactcctggcagacaggggcttccgaaatggtggttgttgacgaggactttgcacct

SEQ ID NO: 95 - DNA, 143 bp, Bos taurus
  misc_feature (72)..(72): n is a, c, g, or t
cagagccgagtaagacagacaatctgagaggcaggagacatcaaaaacttttaggttagaatccatgtatgncttcrgttmttttctgagccggacttcacagttcatggagatcagctgtgattattcgattattttccgtc

SEQ ID NO: 96 - DNA, 143 bp, Bos taurus
  misc_feature (72)..(72): n is a, c, g, or t
ccgagtaagacagacaatctgagaggcaggagacatcaaaaacttttaggttagaatccatgtatgrcttcngttmttttctgagccggacttcacagttcatggagatcagctgtgattattcgattattttccgtctcatt

SEQ ID NO: 97 - DNA, 143 bp, Bos taurus
  misc_feature (72)..(72): n is a, c, g, or t
gtaagacagacaatctgagaggcaggagacatcaaaaacttttaggttagaatccatgtatgrcttcrgttnttttctgagccggacttcacagttcatggagatcagctgtgattattcgattattttccgtctcattaaag

SEQ ID NO: 98 - DNA, 143 bp, Bos taurus
  misc_feature (72)..(72): n is a, c, g, or t
gggggggggggcggtgcagaaggaggcagaccctggtgagtggttccccttgcccttgactttctctctagncagggtaccagccagtggggaccagagccccagtctccccaacagcaccctgatagcacccacataagaca

SEQ ID NO: 99 - DNA, 143 bp, Bos taurus
  misc_feature (72)..(72): n is a, c, g, or t
gccagtggggaccagagccccagtctccccaacagcaccctgatagcacccacataagacacttaccaaagngggggagccgctcggcttccgaggagcagacatgcctctcctgggtcctcctgccgtaggtcgcagagtag

SEQ ID NO: 100 - DNA, 143 bp, Bos taurus
  misc_feature (72)..(72): n is a, c, g, or t
tgggtcctcctgccgtaggtcgcagagtagatgttgagaaacttggactcgtggcagtgcagtttcagctcntggtcttcacacacagttttgttttttaactcatctgaagyagaaaccaaaatcagcggcaccatcagctt

SEQ ID NO: 101 - DNA, 143 bp, Bos taurus
  misc_feature (72)..(72): n is a, c, g, or t
cttggactcgtggcagtgcagtttcagctcrtggtcttcacacacagttttgttttttaactcatctgaagnagaaaccaaaatcagcggcaccatcagcttccccagaaggactcygtgagcattrcccgaatcccaggagg

SEQ ID NO: 102 - DNA, 143 bp, Bos taurus
  misc_feature (72)..(72): n is a, c, g, or t
agttttgttttttaactcatctgaagyagaaaccaaaatcagcggcaccatcagcttccccagaaggactcngtgagcattrcccgaatcccaggaggctcccatcctctgtggggctgcacccccggaaggggacagacggt

SEQ ID NO: 103 - DNA, 143 bp, Bos taurus
  misc_feature (72)..(72): n is a, c, g, or t
tttaactcatctgaagyagaaaccaaaatcagcggcaccatcagcttccccagaaggactcygtgagcattncccgaatcccaggaggctcccatcctctgtggggctgcacccccggaaggggacagacggtgtggccctgc

SEQ ID NO: 104 - DNA, 143 bp, Bos taurus
  misc_feature (72)..(72): n is a, c, g, or t
tgccaggggctctctgaccctgcgagccttctgcggggggcgggggggagcaatgagtggaccaggrtaganmcccagggattacgccaaggaccaaactgaggaggaggagagacgtcagggtgaagaggccagggcgtgct

SEQ ID NO: 105 - DNA, 143 bp, Bos taurus
  misc_feature (72)..(72): n is a, c, g, or t
gccaggggctctctgaccctgcgagccttctgcggggggcgggggggagcaatgagtggaccaggrtagasncccagggattacgccaaggaccaaactgaggaggaggagagacgtcagggtgaagaggccagggcgtgctg

SEQ ID NO: 106 - DNA, 143 bp, Bos taurus
  misc_feature (72)..(72): n is a, c, g, or t
cacacgaggctgggaccccggcatgtctattaataagcgttccgaagctggacggatgccaactggtccacnggcggagcattttcatcaggctatgaatgaaactgggagcccacacccccacgcgctctgccccccttggc

SEQ ID NO: 107 - DNA, 143 bp, Bos taurus
  misc_feature (72)..(72): n is a, c, g, or t
gaccccacgcctgggctgctctggagggacgggcccctgcctgcctggcctggggctcccatttaaagtctnagtctgagcctaagtctaagtctaagacagaagcacctacacggggctgatcggatcggccccggtctgcc

SEQ ID NO: 108 - DNA, 143 bp, Bos taurus
  misc_feature (72)..(72): n is a, c, g, or t
ccagtctccaattagccttgcctggcaccactactgaagttctggcaagagtgacatcgtggaggctcctgnggacataagctatatcaaggtaccacagagggtggactggaagaaacgcagatgcccacccctacccacca

SEQ ID NO: 109 - DNA, 143 bp, Bos taurus
  misc_feature (72)..(72): n is a, c, g, or t
ctctataacgtaggaagagggagacttggcttctctgatctcccactagctgggccaggccttcctggaccnagaggatgcccattttattttagaaaaccagtcctgggcctgcctctgtcctggagaagaaaccaaggcag

SEQ ID NO: 110 - DNA, 143 bp, Bos taurus
  misc_feature (72)..(72): n is a, c, g, or t
actggattgaggttgggagagaatctatgtatgtacacgtatgtgtgtataaatacaaaggtacaggagtcngaggtcatgtttaaattacatttctcactgtgggttaggatcagacaaatttgaaaggggatgttagcaag

SEQ ID NO: 111 - DNA, 143 bp, Bos taurus
  misc_feature (72)..(72): n is a, c, g, or t
tcttttttctactgattggctcctgaccaccttctggatggttaaatggaggaaaatctgaccctggttcantggtatataaagccaactctcaggtttgccaaccatgtttctgcaaactcagagactcactaacatgttat

SEQ ID NO: 112 - DNA, 143 bp, Bos taurus
  misc_feature (72)..(72): n is a, c, g, or t
catgttatttgtaaacgtccaagacagttcccctgtgctgccccaccattcttgggaaagacttgagttacngacttgcatgcycccagccaaagtggaaccagggggatgctctaccctctagtgtcagctcacaaaccaca

SEQ ID NO: 113 - DNA, 143 bp, Bos taurus
  misc_feature (72)..(72): n is a, c, g, or t
aaacgtccaagacagttcccctgtgctgccccaccattcttgggaaagacttgagttacygacttgcatgcncccagccaaagtggaaccagggggatgctctaccctctagtgtcagctcacaaaccacaaacacatgcaaa

SEQ ID NO: 114 - DNA, 143 bp, Bos taurus
  misc_feature (72)..(72): n is a, c, g, or t
cacatgcaaagcacatggtctgtcttgttgagrttcagtcactaagtcatgtccagctccctgcaaccccangaactgcagcatgtcaggcttccctgtccttcactatctgccagagtttgctcaagcttctgtccattgag

SEQ ID NO: 115 - DNA, 143 bp, Bos taurus
  misc_feature (72)..(72): n is a, c, g, or t
ccttcaatctttcccagcatcagggtcttttccaatgagtcagctcttcacatcaggtggccaaagtattanagcttcagtttcagcatcagtccttccgatgaatattcagggttgattttctttaggatagactggtttga

SEQ ID NO: 116 - DNA, 143 bp, Bos taurus
  misc_feature (72)..(72): n is a, c, g, or t
ccctcctgggttctgaaccggtggggccatgctcctgggaaattcagcatcgaaagtagggtctgtgattcngttccgagtgcctgtggggtgtcccagcagagtgtccggagtcagaagctccggagagggacctgggagga

SEQ ID NO: 117 - DNA, 143 bp, Bos taurus
  misc_feature (72)..(72): n is a, c, g, or t
gagaggagacgaagagggagggaagacaggaaggaaggaggaagaggcccagcgtggctgccaaggagatgngagaagtracacaacagccagaggcgagtatcacggggckgagsaaagcgggtgttaccagctctggggac

SEQ ID NO: 118 - DNA, 143 bp, Bos taurus
  misc_feature (72)..(72): n is a, c, g, or t
acgaagagggagggaagacaggaaggaaggaggaagaggcccagcgtggctgccaaggagatgygagaagtnacacaacagccagaggcgagtatcacggggckgagsaaagcgggtgttaccagctctggggaccttgagga

SEQ ID NO: 119 - DNA, 143 bp, Bos taurus
  misc_feature (72)..(72): n is a, c, g, or t
gaagaggcccagcgtggctgccaaggagatgygagaagtracacaacagccagaggcgagtatcacggggcngagsaaagcgggtgttaccagctctggggaccttgaggaaacggtccgacctctctragcctcagatgtgc

SEQ ID NO: 120 - DNA, 143 bp, Bos taurus
  misc_feature (72)..(72): n is a, c, g, or t
aggcccagcgtggctgccaaggagatgygagaagtracacaacagccagaggcgagtatcacggggckgagnaaagcgggtgttaccagctctggggaccttgaggaaacggtccgacctctctragcctcagatgtgcacct

SEQ ID NO: 121 - DNA, 143 bp, Bos taurus
  misc_feature (72)..(72): n is a, c, g, or t
gagtatcacggggckgagsaaagcgggtgttaccagctctggggaccttgaggaaacggtccgacctctctnagcctcagatgtgcacctataaaatgaaggtaacagtaatgacacatctcagrggaccgttgtcaagatta

SEQ ID NO: 122 - DNA, 143 bp, Bos taurus
  misc_feature (72)..(72): n is a, c, g, or t
aaacggtccgacctctctragcctcagatgtgcacctataaaatgaaggtaacagtaatgacacatctcagnggaccgttgtcaagattaactgagctattatgtgtgtagggctcagaactctcctgcattcatttctcayg

SEQ ID NO: 123 - DNA, 143 bp, Bos taurus
  misc_feature (72)..(72): n is a, c, g, or t
grggaccgttgtcaagattaactgagctattatgtgtgtagggctcagaactctcctgcattcatttctcanggcttctgtaacaasgtaccacagactggcrgctgagaagaacagaaatgtattgcctcacagttctggag

SEQ ID NO: 124 - DNA, 143 bp, Bos taurus
  misc_feature (72)..(72): n is a, c, g, or t
gattaactgagctattatgtgtgtagggctcagaactctcctgcattcatttctcayggcttctgtaacaangtaccacagactggcrgctgagaagaacagaaatgtattgcctcacagttctggaggccagaagtccaaaa

SEQ ID NO: 125 - DNA, 143 bp, Bos taurus
  misc_feature (72)..(72): n is a, c, g, or t
atgtgtgtagggctcagaactctcctgcattcatttctcayggcttctgtaacaasgtaccacagactggcngctgagaagaacagaaatgtattgcctcacagttctggaggccagaagtccaaaatcaaggggtggatggg

SEQ ID NO: 126 - DNA, 143 bp, Bos taurus
  misc_feature (72)..(72): n is a, c, g, or t
ataagaacacttatcaaatattggctcttcacatcaggtggccaaagtattggagcttcagcttcagcaacngtccttccaatgaatattcagggttgatttcctttaggattgactggtttgatctccttgcagtccaccag

SEQ ID NO: 127 - DNA, 143 bp, Bos taurus
  misc_feature (72)..(72): n is a, c, g, or t
actggtttgatctccttgcagtccaccagactctcgagagtcttctccagcagcacagttcaaaagcaacanttcggtgctcagccatctttatgggccaattctcacatccatacagaggggaaagaaaacttataatccta

SEQ ID NO: 128 - DNA, 143 bp, Bos taurus
  misc_feature (72)..(72): n is a, c, g, or t
catccatacagaggggaaagaaaacttataatcctaactgttggattagacatctttgtataacaacttcantacttctgtaaagacttaggtaaagattacttctgcaaagactctatctccaaataagggcacaggtacca

SEQ ID NO: 129 - DNA, 143 bp, Bos taurus
  misc_feature (72)..(72): n is a, c, g, or t
gcctgataaggtgatgacattccgcacagaagagtggacaggttcgccctgaagtagcgctagatgattcanccgtgcaatcaacactctcagatccatctttgaacgaaagtctttatataacatgcagtacagagaaggcg

SEQ ID NO: 130 - DNA, 143 bp, Bos taurus
  misc_feature (72)..(72): n is a, c, g, or t
gacaggcctggcgtgctgcagtccatgggtcacaaagagttggacacaacttggtgactgaacaacaacaanacatagaagaggacatgaccttgcatagaaatatcagctcatctatgtgcttgctgtctgtgggtgcaaag

SEQ ID NO: 131 - DNA, 143 bp, Bos taurus
  misc_feature (72)..(72): n is a, c, g, or t
acatagaagaggacatgaccttgcatagaaatatcagctcatctatgtgcttgctgtctgtgggtgcaaagntgggggcgggggtcagagaaggtgcaagaggaacagaaacaggatcagagaaggaggaaagtaggggaagg

SEQ ID NO: 132 - DNA, 143 bp, Bos taurus
  misc_feature (72)..(72): n is a, c, g, or t
atccaagttcacagcatccctgtttgacagctcaagttcaccctctgaaacagttgttaccttcagacagtnccaaacttgggtggtaacctatagactgcttcaaggattgcaaccgcttcactaargaaacaagctctaag

SEQ ID NO: 133 - DNA, 143 bp, Bos taurus
  misc_feature (72)..(72): n is a, c, g, or t
cagcctgcyggtggctgaattcagattgctttccatagcttaaatcagcaaagacagaacttttctagcagncaagaaagttctattaccaacaaaaaaaaagtcaatagcacttgaaattcagcattttttcccagcacatt

SEQ ID NO: 134 - DNA, 143 bp, Bos taurus
  misc_feature (72)..(72): n is a, c, g, or t
ttaccggagaagtccgtgagcgccgcgatctctctggagcaaaccaggacggtgcagtagaacagacgcagnagctgccccggaggctctacctgcctgcggaggccgggatgctgcgcgggttgcggggcggacggtgggcg

SEQ ID NO: 135 - DNA, 143 bp, Bos taurus
  misc_feature (72)..(72): n is a, c, g, or t
cgcccgtgacgcactttggagccagcgccgcgcgctgagccagggagccccggggcggggccgaggccgggntgggggcctcggaaggtggggtccgggaggtgtgggaccgcagcactgacgtctttggcgaccggccgcgc

SEQ ID NO: 136 - DNA, 163 bp, Bos taurus
  misc_feature (72)..(92): n is a, c, g, or t
ccagggacgggggagcctggtgggctgccatctatggggtcacacagagtcggacatgactgaagcgacttnnnnnnnnnnnnnnnnnnnnngtgattgaactgaacccttctctggctgacacctgttacttctgacggccctcagctgacacttgctattc

SEQ ID NO: 137 - DNA, 145 bp, Bos taurus
  misc_feature (72)..(74): n is a, c, g, or t
ggtgcaagaggaacagaaacaggatcagagaaggaggaaagtaggggaaggcagtgggaggaaccaaatctnnncatggttttcaagacttgctgtcttccttttaaaaatcaaattgctcccccgcccaccccatgcccacaac

SEQ ID NO: 138 - DNA, 143 bp, Bos taurus
  misc_feature (72)..(72): n is a, c, g, or t
gtaactggatcaatccccatttgcccaaagcatttacacggaaagctttttcttttctttgggatgcagagnaactcccaaattacagagccacttggcgattaaggcaggatggaggtgcatttgataacttaatgcaactt

SEQ ID NO: 139 - DNA, 138 bp, Bos taurus
  misc_feature (72)..(72): n is a, c, g, or t
ccatgtacaacaaaagcctctcagaaacactcagggaagccgaccaaccaaagggcccaacacagtccaacncawgcatgtgggggcgggccggtgccgcctcccctcgagcccctcaaacagcctttcggctccaga

SEQ ID NO: 140 - DNA, 138 bp, Bos taurus
  misc_feature (72)..(72): n is a, c, g, or t
tgtacaacaaaagcctctcagaaacactcagggaagccgaccaaccaaagggcccaacacagtccaacrcangcatgtgggggcgggccggtgccgcctcccctcgagcccctcaaacagcctttcggctccagacgg

SEQ ID NO: 141 - DNA, 143 bp, Bos taurus
  misc_feature (72)..(72): n is a, c, g, or t
gtagacgtgatacgctcaaggtagaaatgaggacttcagggcatgccttgcgagctttctccagttaattgnaaaattatatagtgatttcactgctcataacaataaaaccttctcttttaactttaaagatgatgatgact

SEQ ID NO: 142 - DNA, 143 bp, Bos taurus
  misc_feature (72)..(72): n is a, c, g, or t
gagcagagaagtttctaaaatctgtaacagatgtctccaagagtctgtccttagggagtccgcgggctgacngactgtatctattacagcctcctgatgggtttaacagaggatgtgggggcaggtcatttagtttccccctt

SEQ ID NO: 143 - DNA, 143 bp, Bos taurus
  misc_feature (72)..(72): n is a, c, g, or t
caaaataaaagcagaggaaaacctgagggggaccatggttatttctttgcatgtgtggttctctcccgcccncatctaaatatttttattaaaaaaaatcatgaaaaaatgttagccccactctatyacctcaatcctagttt

SEQ ID NO: 144 - DNA, 143 bp, Bos taurus
  misc_feature (72)..(72): n is a, c, g, or t
tggttctctcccgcccscatctaaatatttttattaaaaaaaatcatgaaaaaatgttagccccactctatnacctcaatcctagtttttttctattttgaaatggcaaccaggggcacacctttcagccagacaagcactgg

SEQ ID NO: 145 - DNA, 139 bp, Bos taurus
  misc_feature (72)..(72): n is a, c, g, or t
caaacaagagctaaattccaggatggggaggaagaacaaggaaagctagtccacacctcccatcgcccccangccccccttttatttctctctcccactcatgtttttattccacttttctctcctggattggacrtgc

SEQ ID NO: 146 - DNA, 41 bp, Bos taurus
tcttacacatcaggagatagytccgaggtggatttctacaa

SEQ ID NO: 147 - DNA, 41 bp, Bos Taurus; identical to SEQ ID NO: 146

SEQ ID NO: 148 - DNA, 41 bp, Bos Taurus
tcttacacatcaggagatggytccgaggtggatttctacaa